(19) United States
(12) Patent Application Publication
(10) Pub. No.: US 2025/0145967 A1
(43) Pub. Date: May 8, 2025

(54) TISSUE PERFUSION SYSTEM

(71) Applicant: Vascular Perfusion Solutions, Inc., San Antonio, TX (US)

(72) Inventors: Israel Jessop, San Antonio, TX (US); Exal Cisneros, San Antonio, TX (US); Leonid Bunegin, San Antonio, TX (US)

(21) Appl. No.: 18/837,516

(22) PCT Filed: Feb. 9, 2023
(86) PCT No.: PCT/US2023/062281

§ 371 Date: Aug. 9, 2024

Related U.S. Application Data
(60) Provisional application No. 63/267,729, filed on Feb. 9, 2022.

(51) Int. Cl. C12N 5/00

(52) U.S. Cl. CPC C12N 5/542

(57) ABSTRACT

A perfusion system (100) includes a perfusion module (110) configured to circulate a perfusate. The perfusion module includes an oxygenator (112) configured to oxygenate the perfusate, the oxygenator configured to be fluidly coupled with an oxygen source and configured to receive oxygen therefrom, and a first and a second pump, each pump operably coupled with the oxygenator and configured to circulate the perfusate through the oxygenator. The system further comprises a cannister (140) and a tissue interface (150) disposed between the perfusion module and the cannister. The first and second pumps can be diaphragm pumps. The oxygen source can be in fluid communication with the perfusion module to allow flow of oxygen and pressurization of the pump diaphragms (128).

### PRIORITY CLAIM

This application claims the benefit of priority to U.S. Provisional Patent Application Ser. No. 63/267,729, filed Feb. 9, 2022, the contents of which are incorporated herein by reference.

### BACKGROUND

Perfusion includes the passage of fluid through the circulatory system or lymphatic system of an organ or tissue. In the human body, perfusion often refers to passage of blood through a capillary bed in tissue. Perfusion can allow for the delivery of oxygen, other dissolved gases, nutrients, and other items to the tissue. When tissue or an organ is not residing in the body, such as during transport of an organ for transplant, perfusion does not naturally occur, and this can result in unwanted damage to the tissue or organ. Conventional perfusion systems for perfusing an organ outside the body can use an electric pump to circulate the perfusate. These pumps can be large and inefficient.

### SUMMARY OF THE DISCLOSURE

A perfusion system includes a perfusion module configured to circulate a perfusate. The perfusion module includes an oxygenator configured to oxygenate the perfusate. The oxygenator is configured to fluidly couple with an oxygen source to receive oxygen. One or more pumps are operably coupled with the oxygenator and configured to circulate the perfusate through the oxygenator. A cannister is releasably coupled to the perfusion module and includes a chamber or receptacle to receive the target tissue. A tissue interface includes at least one port for mechanically and fluidly coupling the target tissue to the perfusion module.

A method of perfusing target tissue in a perfusion system includes oxygenating perfusate in an oxygenator, pumping the oxygenated perfusate from the oxygenator to a de-pressurized pump chamber, pressurizing the pump chamber and pumping the oxygenated perfusate out of the pump chamber and through a cannula to the target tissue in a cannister, and oxygenating the target tissue.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIGS. 1A, 1B, and 1C are block diagrams of a perfusion system in an example.

FIG. 2 is a cross section view representation of a perfusion system pump in an example.

FIG. 3 is a perspective view representation of a perfusion system pump diaphragm in an example.

FIG. 4 is a block schematic diagram of a perfusion system in an example.

FIGS. 5A-5B are block schematic diagrams of a perfusion system in an example.

FIG. 6 is a chart showing a perfusion system pump pressure over time in an example.

FIGS. 7A-7B are charts of a perfusion system pump pressure over time in an example.

FIGS. 8A-8B are charts of a perfusion system pump pressure over time in an example.

FIGS. 9A-9B are charts of a perfusion system pump pressure over time in an example.

FIG. 10A is a flow diagram illustrating fluid flow in an example.

FIG. 10B is a chart of a perfusion system pump pressure over time in an example.

FIG. 11 is a block diagram representation of a perfusion system tissue interface in an example.

FIG. 12 is a cross section view of a perfusion system connector in an example.

FIG. 13 is a cross section view of a perfusion system connector in an example.

FIG. 14 is a cross section view of a perfusion system in an example.

FIG. 15 is a perspective view of a perfusion system connector in an example.

FIG. 16 is a perspective view of a perfusion system with a dip tube in an example.

FIG. 17 is a block diagram representation of a perfusion system dip tube configuration in an example.

FIG. 18 is a cross section view of a perfusion system with a one-way valve in an example.

FIG. 19 is a block diagram of a perfusion system with a sliding seal in an example.

FIG. 20A-20B are cross section diagrams of a perfusion system two-sided filter chamber in an example.

FIG. 21 is a cross section diagram of a perfusion system with an orifice restrictor in an example.

FIG. 22 is a cross section diagram of a perfusion system with a coronary sinus sample recirculation loop in an example.

FIG. 23 is a cross section diagram of a perfusion system with thermal regulation in an example.

FIG. 24 is a cross section diagram of a perfusion system with phase change materials in an example.

FIG. 25 is a schematic diagram of a perfusion system oxygen flow path in an example.

FIG. 26 is a flow diagram of a perfusion system oxygen flow path in an example.

FIG. 27 is a cross section diagram of a perfusion system in an example.

FIGS. 28A-28B are flow diagrams of a perfusion system having alternative therapeutic gases in an example.

### DETAILED DESCRIPTION

An organ perfusion system used to maintain organ or other tissue viability during transport interval. The system provides perfusate for the organ or other tissue is waiting to be given to the recipient. The system can optionally have an inner lid to better maintain sterility of the device and organ or tissue being transported.

A perfusion system can maintain and prolong organ viability during the transport interval, after removal from a donor but before transplantation into an organ recipient.

Once separated from a living body, organs, limbs, and other vascularized tissues may be oxygenated, and metabolic waste products removed, to maintain viability of the tissues beyond the medically established cold ischemic time. Perfusion can prolong organ viability outside the body. Perfusion systems can pump an oxygen-enriched liquid through the vasculature (e.g., arteries, capillaries, and veins) of tissue. Moreover, perfusion can deliver nutrient gas, such as oxygen, and metabolic substrates, such as glucose, to metabolically active cells and simultaneously remove metabolic waste gas, such as carbon dioxide.

Various features and designs of the perfusion system can facilitate preservation, testing, modification, treatment, or resuscitation of organs by machine perfusion. Organs can be perfused during organ transport or transplant processes, research, and diagnostics, and other ex-vivo organ treatments. The various features and examples discussed herein relate to such ex vivo treatment of organs. Features and examples can include design and control of a perfusion pump, ease-of-use features of a perfusion system, and various operational variations.

Challenges associated with organ or tissue perfusion can include ease of use, maintaining sterile environment, and allowing for fluid-tight seals. Here, a tissue interface is designed and used to connect a perfusion module (such as including an oxygenator and pump) to an organ or tissue within a perfusion cannister.

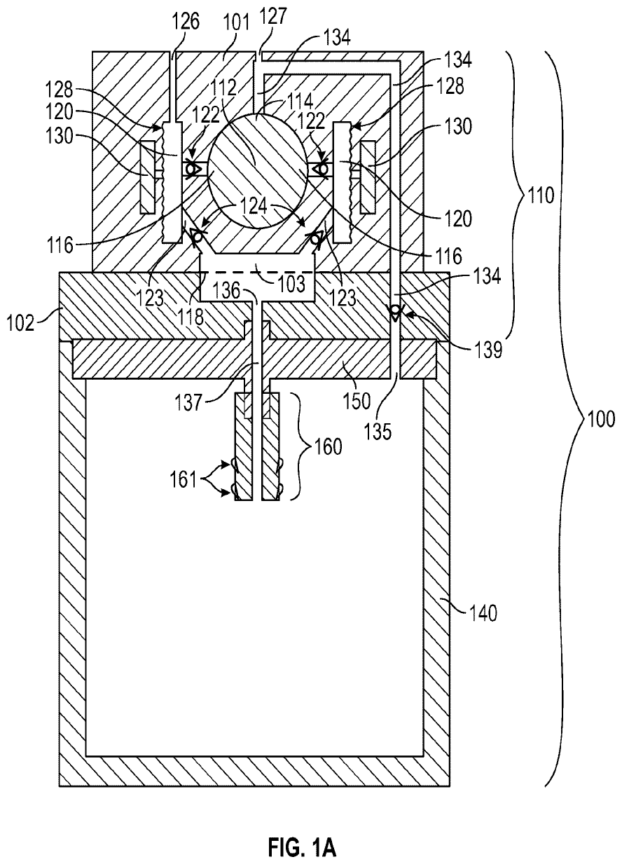

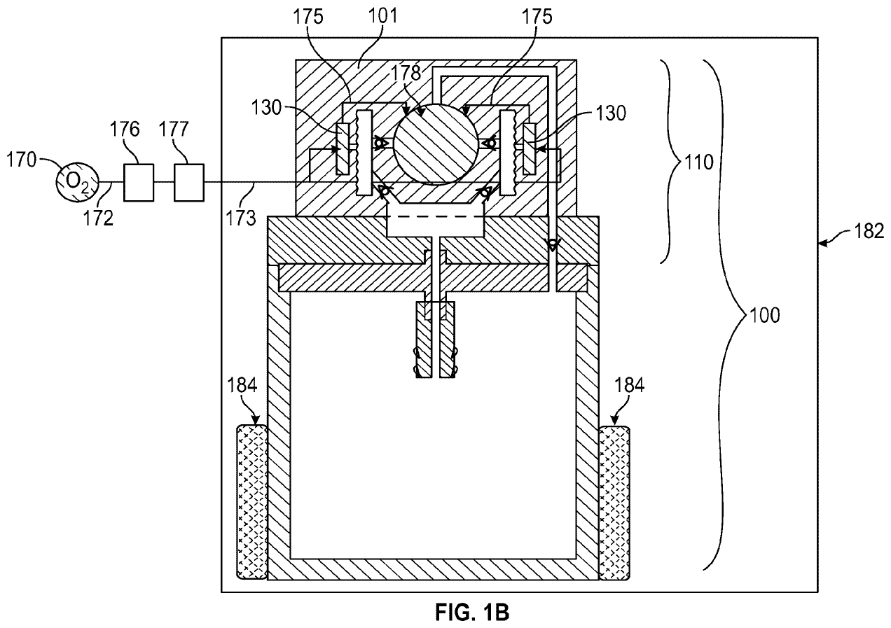

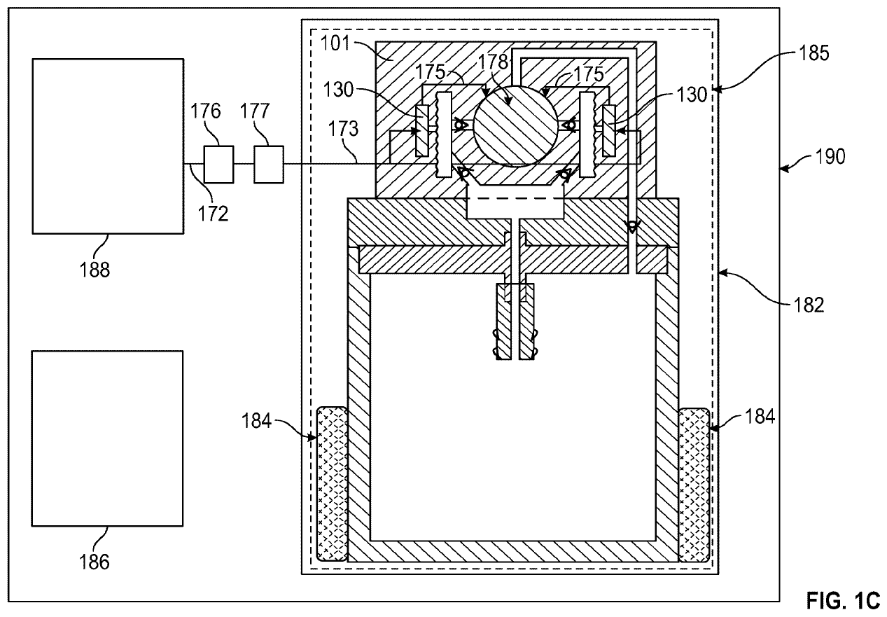

FIGS. 1A, 1B, and 1C illustrate a diagram of a perfusion system 100 in an example. The system 100 can include a perfusion module 110, a tissue interface 150, and a cannister 140. The perfusion module 110 can include pumps, valves, gas exchangers, filters, ports for fluid filling or extraction, sensors, fluid conduits, seals, a manifold 101 to connect the components, a base plate 102, and other components. The tissue interface 150 can include pass-through fluid channels, fluid port mating features, structural supports, and cannula attachment features. The cannister 140 can include a perfusate fluid reservoir, mechanical fasteners, and in some cases, elements for thermal regulation of the system.

In the example system 100, the perfusion module 110 can contain an oxygenator 112, a filter 118, pump chambers 120, and perfusate lines 134 and 136 coming from the cannister 140 and going toward a cannula 160, respectively. In the embodiment shown, two pumps 120 are used in parallel. Other embodiments may utilize a single pump. Throughout the description, elements that are introduced as including one or more of an element may be referred to in the plural form for convenience without precluding examples that include only one of the elements.

In the perfusion module 100, the oxygenator 112 can be disposed within the manifold 101. The oxygenator 112 can include a perfusate inlet 114 and one or more outlets 116. The outlets 116 can provide oxygenated perfusate to one or more pump chambers 120. The filter 118 can be disposed within a filter chamber 103 at the junction between the manifold 101 and the base plate 102, the filter chamber being comprised of cavities in the manifold 101 and/or the baseplate 102. The pump chambers 120 can be positioned to receive perfusate from the oxygenator 112 via the one or more outlets 116. The pump chambers 120 can include inlet valves 122 positioned to control perfusate flow into the pump chambers 120 from oxygenator outlets 116. The pump chambers 120 also can include outlets 123 with valves 124 to control perfusate flow out of the pump chamber into a filter chamber 103 which can connect to a perfusate supply line 136. A vent 126 can be connected to either or both pump chambers 120, pump outlets 123, or filter chamber 103 for venting gas. A vent 127 can connect to the perfusate line 134 for venting gas. The pump chambers 120 can include diaphragms 128 that are coupled to and controlled by valves 130. The diaphragms 128 can be actuated to pump perfusate through the perfusion module 110.

In the example system 100, the cannister can be configured to hold an organ or tissue. The perfusate inlet opening 134 can be fluidically coupled to the cannister 140.

In the example system 100, the tissue interface 150 can be positioned between the canister 140 and the base plate 102 of the perfusion module 110. The tissue interface 150 can include the perfusate inlet opening 135 that fluidly couples to perfusate line 134 and a perfusate line 137 that fluidly couples to perfusate line 136, to which a cannula 160 may be coupled. The cannula 160 can be hermetically sealed with the perfusate line 137 and configured with an end portion 161. The end portion 161 may be configured with one or more barbs or ribs also indicated at 161 to securely couple to an artery of a separated organ to supply oxygenated perfusate via perfusate supply line 136. The tissue interface can provide a secure, fluid tight, connection to cannister 140 and base plate 102 while permitting controlled flow of perfusate to and from the cannister. A one-way valve 139 can be included in perfusate line 134 to prevent retrograde flow of perfusate during priming.

As shown in FIG. 1B, the system 100 can be connected to an oxygen source 170 to supply oxygen via an oxygen line 172. The oxygen line 172 can be coupled to the pressure regulator 176 to regulate the oxygen pressure. The regulator 176 may also be connected to a flow controller or flow restrictor 177 to control or restrict flow of oxygen gas to the system 100. An oxygen supply line 173 can extend from the pump pressure regulator 176 (or flow controller 177 if used) to the valves 130. One or more oxygen supply lines 175 can extend from the valves 130 to the oxygenator 112 to provide oxygen for oxygenation of the perfusate. In one example, the oxygen supply lines 175 can extend or feed through the manifold 101 to reach the oxygenator 112 to ensure proper fluidic sealing of oxygenator 112. The manifold 101 can also include a vent 178 extending from the oxygenator to ambient.

In some cases, such as shown in FIG. 1C, accessories such as a thermal barrier 182, phase change materials 184, electronics module 186, oxygen tank 188, and a carry case 190, can be included.

In the example of FIGS. 1A-1C, the system 100 can be used to circulate perfusate through a target tissue or organ in the cannister 140 to provide oxygen to the tissue. The perfusate can be a perfusate fluid, such a liquid, blood, saline, fluid specifically formulated for organ preservation or perfusion, or some other appropriate fluid for perfusion of an organ or target tissue in the cannister 140. The perfusate fluid can include water, electrolytes, pH buffering components, metabolic substrates, and other ingredients to maintain the viability of the heart. The fluid can be, for example, oxygen-enriched fluid or blood-based fluid, to provide oxygen to the target tissue, organ, or limb. For example, the organ can be a heart, lung, kidney, or other vascular tissue requiring oxygenation while outside the body. The perfusion circuit can include, for example, tubing, piping, or hosing, to carry the perfusate fluid between one or more fluid reservoirs, and the cannister 140.

The perfusion module 110 can include pumps, valves, gas exchangers, filters, ports for fluid filling or extraction, sensors, fluid conduits, seals, and other components. In the example system 100, the perfusion module 110 can house components for circulation of perfusate and oxygen throughout the system 100. The perfusion module 110 can include the oxygenator 112, the filter 118, and the perfusion pump chambers 120, encapsulated by an optional housing (not shown) or a manifold 101. The perfusion module 110 can be connected to the cannister 140, such as through the tissue interface 150. The cannula 160 may fluidly connect the perfusion module 110 to a cannulated organ or target tissue located in the cannister 140 by allowing flow of perfusate therebetween. The oxygen source 170 (FIG. 1B) or 188 (FIG. 1C) can be in fluid communication with the perfusion module 110 to allow flow of oxygen and allow for pressurization of the pump diaphragms 128.

The perfusion module 110 can house the oxygenator 112, the filter 118, the pump chambers 120, and the valves 130. The perfusion module 110 can include an optional housing for encapsulating or covering the components, such as a metallic, composite, or plastic material, for at least partially enclosing and protecting the components in the perfusion module 110. The perfusion module 110 can be shaped, sized, or arranged for optimal layout of the components in the perfusion module 110 while allowing for pumping of perfusate and oxygen through the system 100.

The oxygenator 112 can be configured to exchange oxygen and carbon dioxide in perfusate fluid. The oxygenator 112 can include a perfusate inlet 114 for incoming de-oxygenated perfusate from the cannister 140, and perfusate outlets 116, wherein outgoing oxygenated perfusate can exit the oxygenator 112. The oxygenator 112 can be secured within the perfusion module 110, such as to a base plate 102, or within a manifold 101.

The oxygenator can be fluidly coupled to the oxygen source 170 (as in FIG. 1B) or 188 in FIG. 1C. The oxygen source 170 or 188 can be an oxygen concentrator, an oxygen generator, tank of pressurized oxygen, or other appropriate oxygen source, such as a hook-up. The oxygen source 170 can provide oxygen to the organ preservation system 100 and provide a pressure gradient to the system 100 to induce flow of a perfusate fluid therethrough. The oxygen source 170 or 188 may also supply oxygen in mixture with other gases, such as carbogen (95% oxygen/5% carbon dioxide, oxygen/nitrous oxide mixtures, oxygen/hydrogen mixtures, etc.)

For example, the oxygen source 170 can be an oxygen concentrator that can filter surrounding air, compress that air to a specified density, and deliver purified oxygen in a pulsatile fashion, or in a continuous stream. Such an oxygen concentrator can be fitted with filters and/or sieve beds to remove nitrogen and other elements, gases, or contaminants from the air. In an example, the oxygen concentrator can include a pressure swing adsorption system, such as the Invacare® Platinum Mobile oxygen concentrator (Invacare Corporation, Elyria, OH). A pressure swing adsorption oxygen concentrator can leverage a molecular sieve to absorb gases and operate using rapid pressure swing adsorption to capture atmospheric nitrogen in minerals, such as zeolite, and subsequently vent that nitrogen, operating in a manner that is similar to a nitrogen scrubber. This can allow other atmospheric gases to exit the system, leaving oxygen as the primary remaining gas. Conventional oxygen concentrators can include an air compressor, the molecular sieve or alternatively a membrane, a pressure equalizer, and various valves and tubes to accomplish these functions. Other types or configurations of oxygen concentrators or oxygen sources are also envisioned herein.

In some cases, the oxygen source 170 can be an oxygen generator. In this context, an oxygen generator can produce molecular oxygen (O₂ gas) by reaction of other chemical components. Examples of oxygen-generating chemical reactions can include thermal decomposition of chlorate or perchlorate salts, hydrolysis of potassium superoxide, enzyme (catalase)-mediated decomposition of hydrogen peroxide, electrolysis of water, or other appropriate reactions.

The pressure of the oxygen provided by the oxygen source 170 can be regulated by pump pressure regulator 176. The pressure can be about, for example, 75 mm Hg. Also waste gas can be vented out of the oxygenator at vent 178.

In the oxygenator 112, de-oxygenated perfusate fluid from the cannister 140 can enter through the inlet 114. The perfusate can run up through the oxygenator towards the outlets 116. While in the body of the oxygenator, the perfusate can be oxygenated. For example, the oxygenator 112 can be a hollow cylinder with a central lumen that the perfusate runs through. The cylinder of the oxygenator can include one or more structures or components that allow for dissolution of oxygen within the perfusate. The oxygen and the perfusate within the oxygenator 112 can run in directions opposite each other, to create a counter-current flow. Such a counter-current flow can increase the gradient and the oxygenation of the perfusate by diffusion of oxygen gas therein.

The filter 118 can be, for example, a plate filter across the junction of the manifold and base plate 102, so that oxygenated perfusate leaving the pump chambers 120 can be filtered for impurities before being cycled back towards the cannula (160) and attached organ or tissue. The filter chamber 103 formed by the combination of a cavities in the manifold 101 and base plate 102 where they come together.

The filter can include, for example, a particulate filter, a filter for removing contaminants in the perfusate fluid, a filter directed to chemicals or dissolved gases, or any other type of appropriate filter for treatment of the perfusate fluid. In any example of the portable oxygen source and perfusion system disclosed herein, multiple filters can be used. In some cases, a filter can be upstream of the tissue container of the organ preservation system 100 so as to filter the perfusate fluid prior to reaching the tissue or organ being perfused. In some cases, the filter can be downstream of the tissue container of the organ preservation system 100 so that fluid returning to the tissue container reservoir is filtered.

The oxygenated perfusate can flow out of the oxygenator 112 through the valves 122 into the pump chambers 120. The pump chambers 120 can have inlet valves 122 and outlet valves 124, which can be check valves. The diaphragms 128 in the pump chambers 120 can be de-pressurized to allow flow of the oxygenated perfusate into the pump chamber 120s. The oxygenated perfusate can flow into the pump chambers 120 through the inlet valves 122, and fill the pump chamber 120s partially or fully. The oxygenated perfusate can remain in the pump chambers 120 until it is pumped out towards the filter 118 and cannula 160.

The diaphragms 128, located in the pump chambers 120, can be pressurized to pump perfusate out of the pump chambers 120, through the outlet valves 124, and towards the target tissue in the cannister 140 via line 136. Articulation of the diaphragms 128 can allow pumping of the perfusate out of the pump chambers 120.

The valves 130 can be controllable solenoid valves situated in the oxygen line 173 between the oxygen pressure regulator 176 (or flow restrictor/regulator 177) and the oxygenator 112. The valve 130 is also between line 173 and diaphragms 128. Valves 130 may be fluidly coupled to the diaphragms 128.

The cannister 140 can include a perfusate fluid reservoir, mechanical fasteners, and in some cases, elements for thermal regulation of the system. In the example of system 100, the cannister 140 can be a container for the target tissue or organ being perfused. For example, the cannister 140 can contain the perfusate and a heart (or other organ or tissue), coupling with the perfusion module 110 to form a sterile barrier around the organ, enclosing it within a fluid-tight container. The cannister 140 can provide a sterile environment in which to transport and perfuse the target tissue and organ; the cannister 140 can be filled with a perfusate in which the target tissue or organ resides.

The tissue interface 150 can include pass-through fluid channels, fluid port mating features, structural supports, and cannula attachment features. In the example system 100, the cannister 140 can create a seal with the tissue interface 150, and be fluidly connected to the components of the perfusion module 110 through the cannula 160 and the tissue interface 150. The seal can be created by attachment mechanisms, such as threading, a snap fit, a press fit, O-rings, or other sealing attachments to allow for a liquid-tight seal. In the example system 100, the perfusion head can be held in place atop the cannister 140, such as by buckles or latches.

The tissue interface 150 between the cannister 140 and the perfusion module 110 can separate the two. The tissue interface 150 can additionally mediate fluid transport between the perfusion module 110 and the target tissue or organ, and back into the perfusion module 110.

The cannula 160 can allow for a cannula to fluidly connect the perfusion module 110 to the target tissue through the tissue interface 150. For example, where a heart is being transported and perfused, the cannula 160 can include a cannula that can fluidly couple the aorta of the donor heart to the output of the head unit, and also support the weight of the donor heart during transfer to the sterile surgical field.

In some cases, such as shown in FIG. 1B, accessories such as a thermal barrier 182 configured to enclose the system 100 to prevent heat transfer to and from an ambient environment may be included. Phase change materials 184 can be coupled to container 140. An electronics module 186 and, oxygen tank 188 and thermal barrier 182 may be disposed or otherwise supported within a carry case 190 for convenient transport.

The thermal barrier 182 and the phase change materials 184 can be used to insulate the system 100. The electronics module 186 can be electrically coupled to the perfusion system 100, such as to provide power, and allow connection of the system 100 to a user interface. The oxygen tank 188 can be fluidly connected to the system 100 and provide oxygen gas for perfusion of tissue. The carry case 190 can allow for movement of the system 100, such as during organ transport.

A sterile bag or flexible enclosure 185 may be interposed between the perfusion system 100 and the thermal barrier 182, as shown in FIG. 1C. The sterile bag may be used to preserve sterility of the outer surfaces of the perfusion system 100 during transport within the thermal barrier 182 and outer case 190.

### Perfusion Pumps

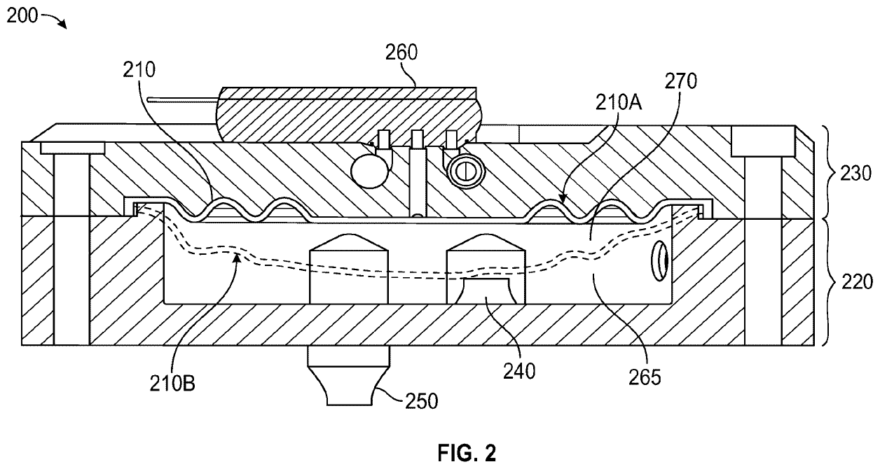

FIGS. 2 to 6 relate to types and function of perfusion pumps, amounts and arrangement of perfusion pumps, and duty cycles of perfusion pumps, such as can be used in a perfusion system.

FIG. 2 illustrates a diagram of a perfusion system pump 200 in an example. The pump 200 can include a diaphragm 210, pump body 220, pump cover 230, an inlet valve 240, an outlet valve 250, and a valve 260. The diaphragm 210 can be at least partially enclosed and sealed between the pump body 220 and the pump cover 230 creating a pumping chamber 265 bounded by the diaphragm 210 and pump body 220. The valve 260 can allow for movement of the diaphragm and pumping of perfusate in and out of the valves 240, 250 by changing the volume of pumping chamber 265.

The diaphragm 210 can be an elastic barrier between the pressurized gas and the perfusate in a perfusion system. The diaphragm 210 can be alternatively pressurized and de-pressurized to actuate the pump 200. Thus, articulation of the diaphragm 210 can allow pumping of the perfusate. Shown in FIG. 2, the diaphragm 210 can be in a first configuration 210A or a second configuration 210B. In the first configuration 210A, the diaphragm can be relaxed. Moving to the relaxed position causes fluid flow into pumping chamber 265 via inlet valve 240. In the second configuration 210B, the diaphragm can be pressurized, reducing the volume of pumping chamber 265 causing flow out of outlet valve 250.

The pump body 220 and pump cover 230 can help secure, stabilize, and protect the diaphragm within the system. The inlet valve 240 can allow for fluid to enter the pump body 220 and pressurize the diaphragm 210. The outlet valve 250 can allow for fluid to exit the pump body 220 and de-pressurize the diaphragm 210. The solenoid valve 260 can serve to actuate the pump 200.

The pump body 220 can include a cavity which is bounded on one side by the diaphragm 210. The pump body can fill with perfusate when the diaphragm 210 is relaxed (e.g., not pressurized by gas), and the perfusate can be forced out of the pump 200 by the diaphragm 210 when the diaphragm 210 is pressurized by gas.

The inlet valve 240 and outlet valve 250 in the pump body 220 can be one-way valves that control the direction of perfusate flow through the pump. The pump cover 230, and diaphragm 210, can define the volume, chamber 270, into which the gas expands during the pressurization cycle. The solenoid valve 240 can direct the compressed gas flow through the pump cover 230, either towards the diaphragm 210 (e.g., for pressurization) or away from the diaphragm 210 (e.g., for gas venting).

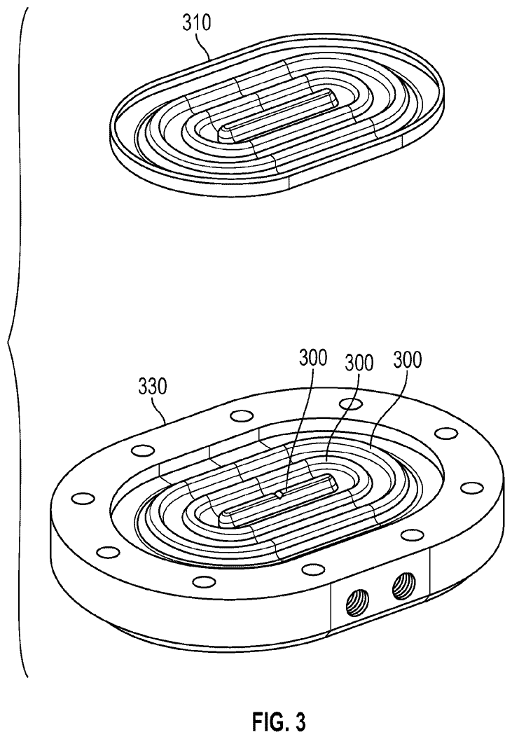

FIG. 3 illustrates a diagram of components of a perfusion system pump 300 in an example. The pump components 300 can include a diaphragm 310 and a pump cover 330. The use of convolutions in the diaphragm 310 and pump cover 330 can help reduce non-working gas volume, e.g., space between the diaphragm and the pump cover when the diaphragm is relaxed. In one example, the convolutions comprise multiple nested racetrack shaped, oval, or circular convolutions.

The diaphragm 310 can be configured with convolutions, such as to minimize elastic resistance to deflection. This can help reduce pressure differences between the driving pressure (e.g., the compressed gas) and resultant perfusion pressure. The convolutions correspond to convolutions in the pump cover 330. As the convolutions are matched, non-working gas volume can be reduced.

In some cases, valves can be mounted directly to the pump cover 330, such as to reduce non-working gas volume between the diaphragms 310 and valves. This can help reduce non-working gas volume and help energy efficiency of the pump 300.

The example pumps 200, 300, can be used in any of the perfusion systems discussed herein, such as with the system 400 and 500 discussed below, which leverage two pumps each.

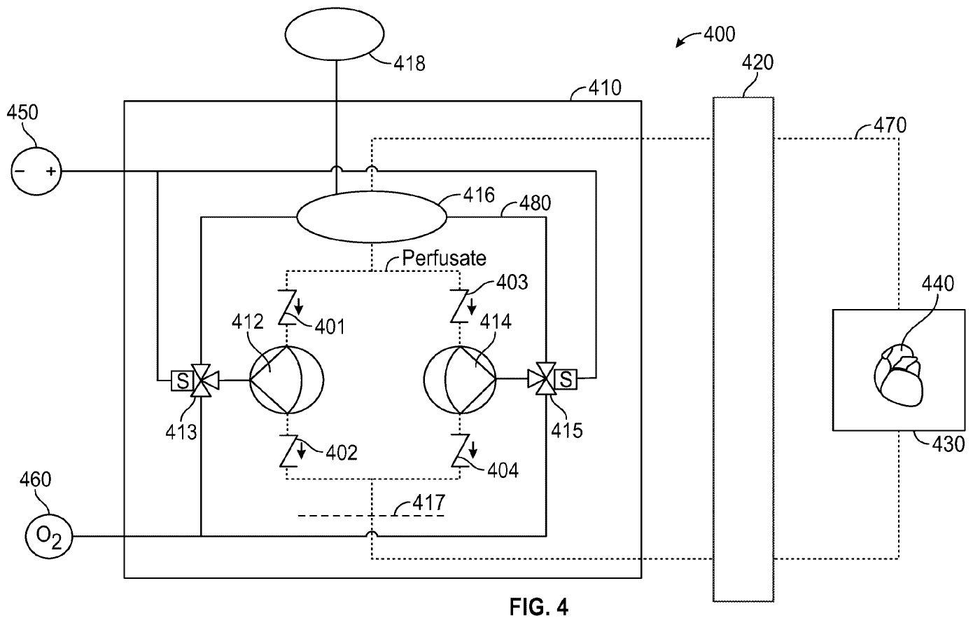

FIG. 4 illustrates a schematic diagram of a perfusion system 400 in an

example. The system 400 can include a perfusion module 410, a tissue interface 420, and a cannister 430 for a heart 440. The perfusion module 410 can include first pump 412, valve 413, second pump 414, valve 415, oxygenator 416, filter 417, and vent 418. One-way valves 401 and 402 can be situated upstream and downstream respectively, of first pump 412. One-way valves 403 and 404 can be situated upstream and downstream, respectively of the second pump 414. The system 400 can additionally include a power source 450 and an oxygen source 460. In the system 400, perfusate 470 and oxygen 480 can flow.

The perfusion module 410 can allow for perfusion of tissue in the system 400. The perfusion module 410 can include two pumps: first pump 412 and second pump 414. The first pump 412 can be fluidly coupled to the first valve 413, while the second pump 414 can be fluidly coupled to the second valve 415. The valves 413 and 415 are controlled by electrical pulses from the power source 450. The pumps 412, 414, can be, for example, similar to the pumps 200, 300, discussed above.

The oxygenator 416 can be configured to exchange oxygen and carbon dioxide in perfusate fluid. The oxygenator can include a perfusate inlet for incoming oxygen-depleted perfusate, and a perfusate outlet, wherein outgoing oxygen-enriched perfusate can exit the oxygenator 416. The oxygenator 416 can be secured within the perfusion module 410, such as within a manifold or to a base plate.

The filter 417 can include, for example, a particulate filter, a filter for removing contaminants in the perfusate fluid, a filter directed to chemicals or dissolved gases, or any other type of appropriate filter for treatment of the perfusate fluid. In any example of the portable oxygen source and perfusion system disclosed herein, multiple filters can be used. In some cases, a filter can be upstream of the cannula of the organ preservation system so as to filter the perfusate fluid prior to reaching the tissue or organ being perfused. In some cases, the filter can be downstream of the tissue container of the organ preservation system so that fluid returning from the tissue container reservoir is filtered. The vent 418 can allow for venting of gas from the system 400 when desired.

The tissue interface 420, can include pass-through fluid channels, fluid port mating features, structural supports, and cannula attachment features. For example, the cannister 430 can create a seal with the tissue interface 420 and be fluidly connected to the components of the perfusion module 410, such as through a cannula. The seal can be created by attachment mechanisms, such as threading, a snap fit, a press fit, O-rings, or other sealing attachments to allow for a liquid-tight seal.

The cannister 430 can include a perfusate fluid reservoir, mechanical fasteners, and in some cases, elements for thermal regulation of the system. For example, the cannister 430 can be a container for the target tissue or organ being perfused. For example, the cannister 430 can contain the perfusate and a heart (or other organ or tissue), coupling with the perfusion module 410 or tissue interface 420 to form a sterile barrier around the organ, enclosing it within a fluid-tight container. The cannister 430 can provide a sterile environment in which to transport and perfuse the target tissue and organ; the cannister 430 can be filled with a perfusate in which the target tissue or organ resides.

The system 400 can additionally include a power source 450 and an oxygen source 460. The power source 450 can be electrically coupled to the system 400 to allow for power thereto. The power source 450 can be, for example, a portable power source such as a battery system. The power source can include a timing mechanism for cycling the power between valves 413 and 415 in a periodic fashion. The oxygen source 460 can be in fluid communication with the perfusion module 410 to allow flow of oxygen, and allow for pressurization of the pumps 412 and 414 of the perfusion module 410. The oxygen source 460 can be, for example, an oxygen tank or other suitable oxygen source.

In the system 400, perfusate 470 and oxygen 480 can flow along a fluid circuit as pressurized by the two pumps 412, 414. In the system 400, the two pumps 412, 414, can be the same or similar types of pumps. In the example of the system 400, the pumps 412, 414, can be arranged parallel each other in the fluid circuit. The pumps 412, 414 can be driven by compressed oxygen from the oxygen source 460, regulated to a pressure between about 25 mm Hg and 500 mm Hg. The pumps 412, 414 can function by using the compressed oxygen gas to move a flexible diaphragm (such as the diaphragms discussed above with reference to pumps 200 and 300), which can in turn pressurize the liquid perfusate and causes it to flow out of the pump and through the rest of the system. In this example, the perfusate can pass from the pumps 412, 414, to the filter 417, out of the perfusion module 410, and through the tissue interface 420 to the heart 440, which can be positioned within the cannister 430. Perfusate can exit from the heart 440 to the cannister 430, and then back into the perfusion module 410. Upon reentering the perfusion module 410, the perfusate stream can pass through the oxygenator 416 before arriving back at the pumps 412, 414, to complete the fluid circuit.

In some examples, the perfusate stream can pass through the oxygenator, pumps, and filter in a different sequential order. The two pumps 412, 414 in parallel can allow for reciprocating fluid flow within the system 400.

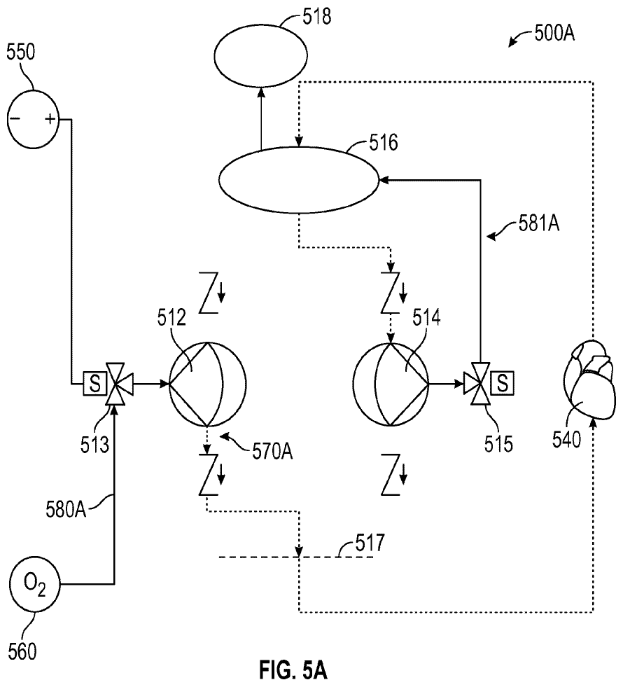

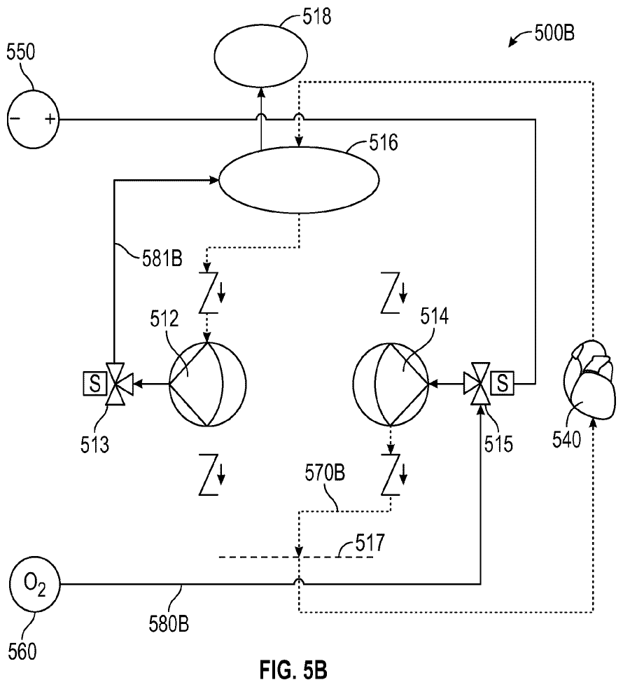

FIGS. 5A-5B illustrate schematic diagrams of a perfusion system 500 with two pumps in an example, having two alternating and complementary operational states represented separately by FIG. 5A and FIG. 5B respectively. Only the fluidic connections having flowing fluid are shown in each of the different states. The system 500 can include a first pump 512, valve 513, second pump 514, valve 515, oxygenator 516, filter 517, and vent 518. The system 500 can additionally include a power source 550 and an oxygen source 560. The components of system 500 can be similar to those of system 400 above, except where otherwise noted. FIG. 5A and FIG. 5B depict alternating fluid flow paths.

The dual pump configuration of system 500 can provide for redundancy in the event of single pump (e.g., pump 512, or pump 514) failure, and can provide for improved efficiency compared to a single pump design.

In the system 500, overall pump efficiency can be improved by splitting the pumping work between two separate diaphragm pumps 512, 514, such that the gas pressurization cycle of the first pump 512 coincides with the gas venting cycle of the second pump 514, and vice versa. The gas pressurization and venting cycles can correspond respectively to the cycles of: (a) perfusate being forced out of the pump outlet in the direction of the filter and heart; and (b) perfusate from the oxygenator flowing through the pump inlet to refill or prime the pump for the next pressurization cycle.

In FIG. 5A, the system 500A depicts the state in which pump 512 is pressurized and pump 514 is venting. The power source 550 activates valve 513, causing pressurized oxygen 580A from the source 560 to flow to the pump 512. Pressurization of pump 512 causes perfusate 570A to flow from the pump 512 outlet through the filter 517 and towards the heart 540. From the heart 540 the perfusate flows through the oxygenator 516 and into pump 514 which is not pressurized. Valve 515 is deactivated, therefore pressurized oxygen 581A from pump 514 is vented through the oxygenator 516 to the ambient vent 518.

In FIG. 5B the system 500B depicts the state in which pump 514 is pressurized and pump 512 is venting. The power source 550 activates valve 515, causing pressurized oxygen 580B from the source 560 to flow to the pump 514. Pressurization of pump 514 causes perfusate 570B to flow from the pump 514 outlet through the filter 517 and towards the heart 540. From the heart 540 the perfusate flows through the oxygenator 516 and into pump 512 which is not pressurized. Valve 513 is deactivated, therefore pressurized oxygen 581B from pump 512 is vented through the oxygenator 516 to the ambient vent 518.

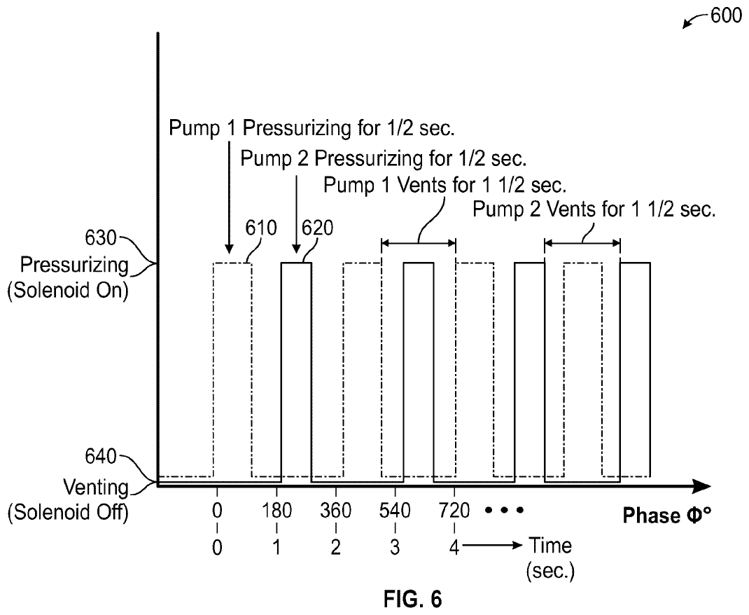

FIG. 6 is a chart 600 of a perfusion system pump pressure in an example. The chart 600 depicts phase on the x-axis and the gas pressure on the y-axis as controlled by the valves depicted as 130 (FIG. 1), 260 (FIGS. 2), 413 and 415 (FIGS. 4), and 513 and 515 (FIGS. 5A and 5B). Pressurizing phases (P) are opposed to venting (V) phases. The first pump and the second pump work in opposition.

FIG. 6 illustrates pressurization and de-pressurization of a system with two pumps, such as systems 400 or 500 above. FIG. 6 depicts examples of pump duty cycle parameters. In FIG. 6, dashed line 610 represents a first pump (e.g., pump 512) cycle, while solid line 620 represents a second pump (e.g., pump 514) cycle. Pressurization periods for both pumps can be about 0.5 seconds. Venting period for both pumps can be about 1.5 seconds.

The pump output can be regulated by regulation of the driving gas pressure, or by electronic regulation of the pump duty cycle. Since the pumps are pressurized and vented according to the position of their controlling solenoid valves, (e.g., solenoid valve 260 above) an adjustment to the cycle of solenoid opening and closing, represented on the y-axis by positions 630 and 640 respectively, can be used to adjust the total pump output.

The duty cycle can be modified by several different methods. In a first example, the time duration of the pressurization cycle can be increased or decreased to achieve a corresponding increase or decrease in stroke volume. In a second example, the length of the venting cycle can be either increased or decreased to achieve a decrease or increase (respectively) in the stroke rate of the pump. Thus, total pump output can be increased by increasing either stroke volume or stroke rate (or both), and total pump output is decreased by reducing either stroke volume or stroke rate (or both.)

Benefits of pump regulation by duty cycle include simplification of the compressed gas flow path by elimination of the variable gas pressure regulator (e.g., upstream of the solenoid valves), as well as the ability to control the pump output by electronic feedback and control loop. For example, a low reading of perfusion pressure or flow rate could trigger an automatic change in the solenoid duty cycle to increase pump output.

### Aortic Valve Closure

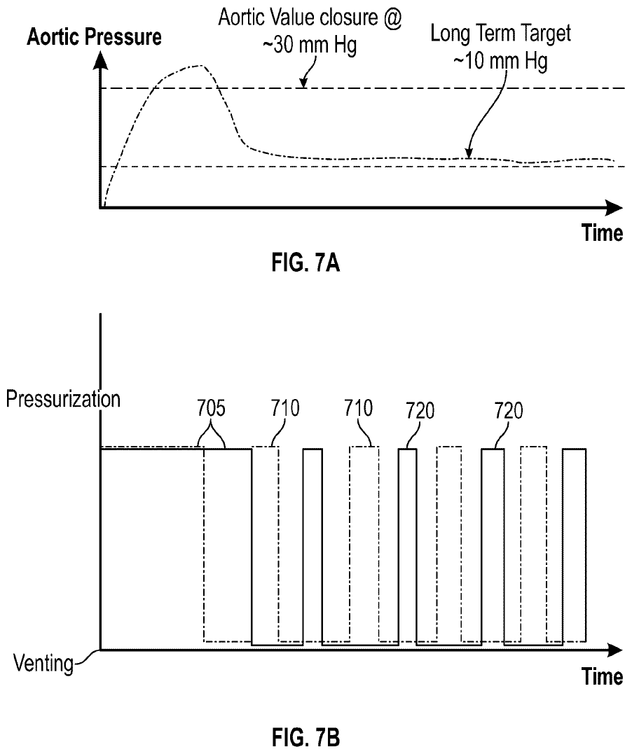

FIGS. 7A to 10B below relate to confirmation of aortic valve closure within a perfusion system, such as system 100 discussed above.

Upon initiation of pumping, the pump can be configured to deliver an initially high flow rate (also referred to as a “bolus”) of perfusate prior to settling into a lower target flow rate. This can be desirable for aortic perfusion of hearts because a high aortic pressure is desired initially in order to close the aortic valve. Closure of the aortic valve helps ensure adequate perfusion of the coronary arteries. An open or partially open aortic valve can allow perfusate volume to bypass the coronary arteries via the left heart chambers, leaving the heart without adequate perfusion.

A gradual buildup of aortic pressure from static (also referred to as “zero flow”) conditions can be insufficient to effectively close the aortic valve. For example, an open aortic valve may occur if the aortic pressure is increased directly from zero up to a target perfusion pressure of about 10 mm Hg. However, closure of the aortic valve can be assured by initially pressurizing the aorta to higher pressures, such as 30 mm Hg or more, for example. After initial pressurization and closure of the aortic valve by higher pressure, the aortic pressure can be gradually reduced down to the long-term target pressure, such as 10 mm Hg, for example, without risk of aortic valve opening. The methods shown and discussed with reference to FIGS. 7A to 10B below can leverage such a brief period after pump startup, after which normal operation of the pump ensues, to allow closure of the aortic valve.

FIGS. 7A-7B illustrate charts of a perfusion system pump pressure in an example. In FIG. 7A, aortic pressure on the y-axis is graphed against time on the x-axis. In FIG. 7B, venting and pressurization of the two pumps (e.g., solenoid valve 260 above) is shown on the y-axis against time on the x-axis. Pressurization cycle of the first pump is indicated by the dashed line 710, and pressurization cycle of the second pump is indicated by the solid line 720.

In FIGS. 7A to 7B, a method of allowing proper closure of the aortic valve in a perfusion system can be accomplished by delivering an initial aortic valve closing bolus. This bolus can be delivered by simultaneous pressurization of two diaphragm pumps 705 for an initial period of, for example, two seconds. This can be followed by a transition to a 180-degree phase offset between the two pumps.

Here, the initial burst of pressure from the bolus can close the aortic valve, after which the aortic pressure can gradually decay to a targeted perfusion pressure. In FIG. 7A, the overall aortic pressure versus time is shown. In FIG. 7B, pressurization and venting of the first pump and the second pump versus time are graphed separately, showing the phase offset of the two pumps after initial bolus.

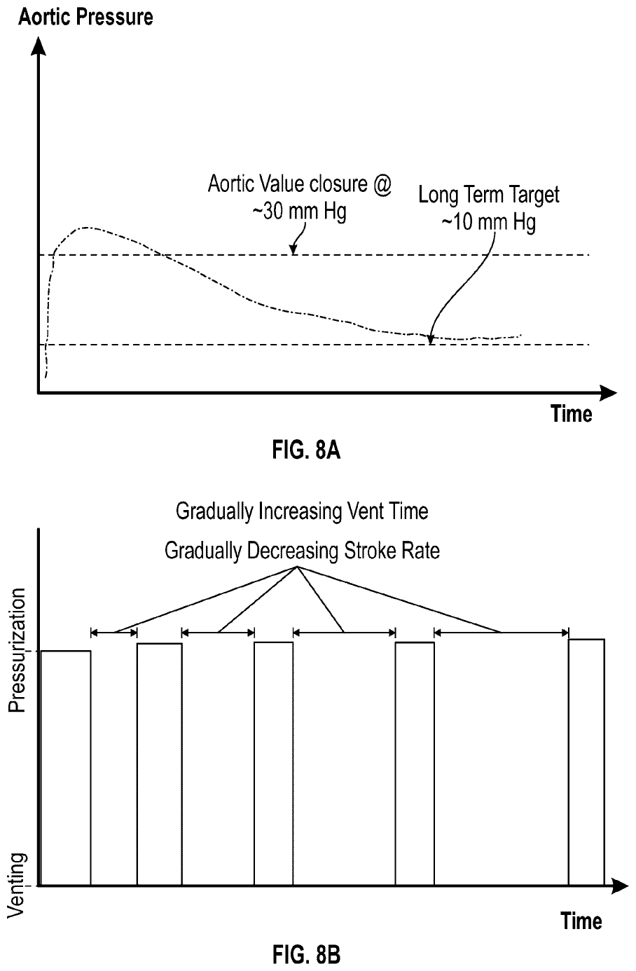

FIGS. 8A-8B illustrate charts of a perfusion system pump pressure in an example. In FIG. 8A, aortic pressure on the y-axis is graphed against time on the x-axis. In FIG. 8B, venting and pressurization of one or more pumps is shown on the y-axis against time on the x-axis.

In FIGS. 8A to 8B, a method of allowing proper closure of the aortic valve in a perfusion system can be accomplished by delivering an initial aortic valve closing bolus by a temporarily elevated pump stroke rate. The initially high stroke rate can provide sufficient pump output to assure aortic valve closure. After a pre-programmed time, interval, such as for example, 4 seconds, the pump stroke rate can be reduced to a steady target. Correspondingly the aortic pressure can decay to target perfusion pressure (see FIG. 8A). In FIG. 8B, a composite pressurization/venting duty cycle of the first pump and the second pump combined is represented with gradually decreasing stroke rate and gradually increasing vent time represented over time.

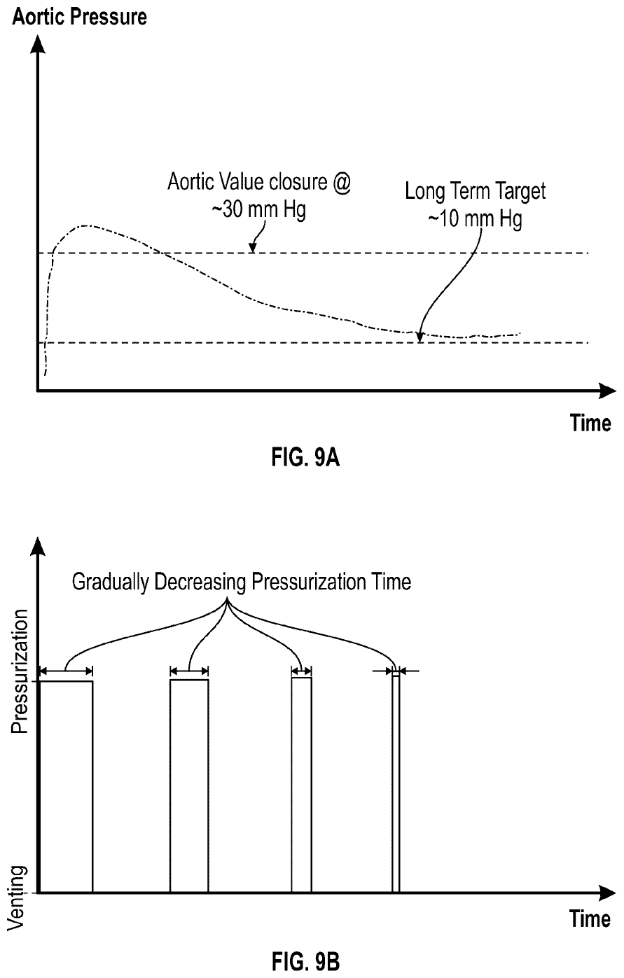

FIGS. 9A-9B illustrate charts of a perfusion system pressures in an example. In FIG. 9A, aortic pressure on the y-axis is graphed against time on the x-axis. In FIG. 9B, venting and pressurization of one or more pumps is shown on the y-axis against time on the x-axis.

In FIGS. 9A and 9B, the aortic valve closure can be achieved by a temporarily extended gas pressurization time to produce a corresponding temporary boost in pump stroke volume. In FIG. 9B, the red line represents the composite duty cycle of the first pump and the second pump combined.

In an example, the methods of pump output control depicted in FIGS. 7, 8, and 9 can be used separately or in combination to control output of the perfusion pump. The control methods can apply to a single diaphragm pump system, or to a dual diaphragm pump system as described in FIGS. 1A, 1B, 1C, 4, 5A, and 5B. In another example, the pump control methods are controlled by a PID automated feedback and control loop to maintain perfusion pressure with respect to a desired target, based on feedback data from perfusion pressure sensors.

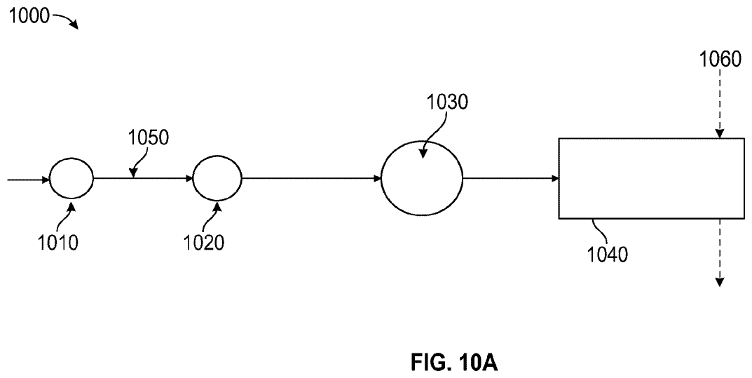

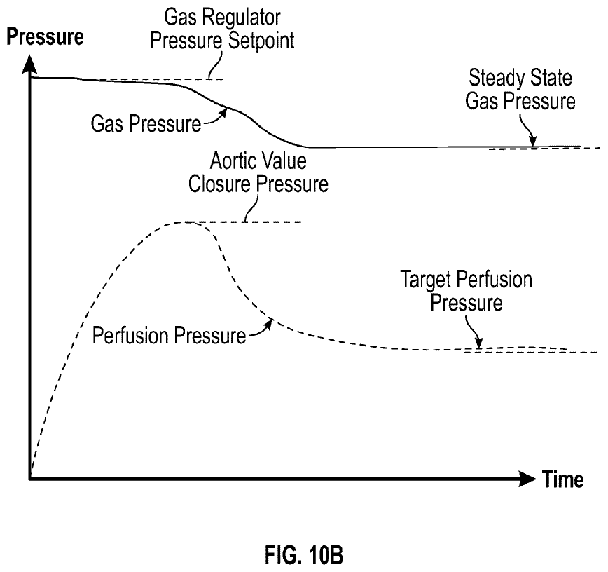

FIGS. 10A-10B illustrate charts of a perfusion system pump pressure in an example. FIG. 10A depicts a flow chart of oxygen and perfusate flow in a system 1000, including a pressure regulator 1010, a flow-restricting element 1020, an accumulator 1030, a pump 1040, with oxygen flow 1050 and perfusate flow 1060. FIG. 10B graphs gas pressure feeding into the pumps and perfusion pressure coming out of the pumps on the y-axis against time on the x-axis.

In the system 1000, the pressure regulator 1010 can be a component for increasing or decreasing pressure of the oxygen. The flow-restricting element 1020 can be a valve or other component for reducing or slowing flow of the oxygen gas. The accumulator 1030 can be for collecting a desired amount of oxygen flow. The pump 1040 can be a single pump or two pumps. In the single pump example, similar PID or other pump control methods may be used to obtain the desired aortic pressure curves.

In FIGS. 10A and 10B, the initial aortic valve-closing bolus can be delivered by a temporarily elevated gas pressure acting on the pump diaphragms. In a gas flow-restricted configuration, a gas accumulator in the compressed gas pathway can act automatically as an initial boost to pump output. After reaching valve-closing pressure, the gas flow can reach a steady state determined by the gas flow restricting elements, causing the pump output to decay and settle to a lower target pressure.

### Perfusion System Connectors

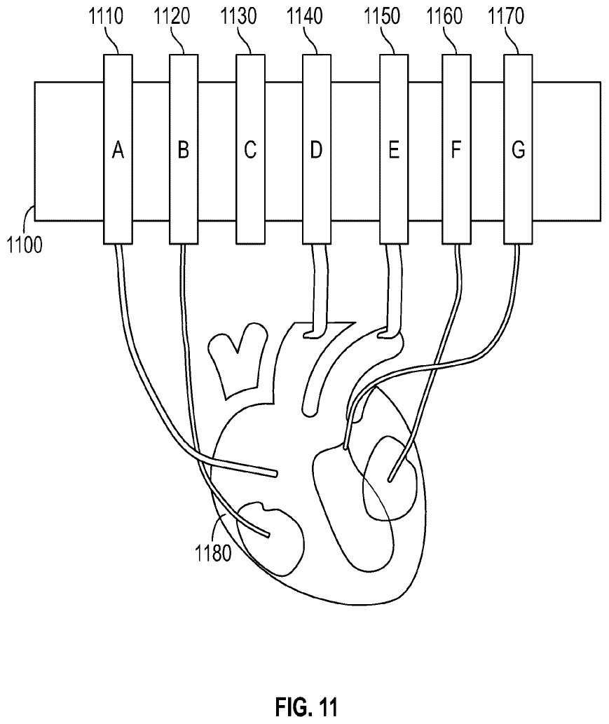

FIGS. 11 to 15 below discuss various types of connectors that can be used in or with a perfusion system, such as to connect a heart, other organ, or tissue, to the system for perfusion.

FIG. 11 illustrates a diagram of a perfusion system tissue interface 1100 in an example. The tissue interface 1100 can include a coronary sinus sampling cannula 1110, a first EKG electrode 1120, a pass-through 1130, an aortic perfusion cannula 1140, a left atrial perfusion cannula 1150, a second EKG electrode 1160, and a left ventricle balloon catheter 1170. A heart 1180 can be attached to the tissue interface 1100.

The tissue interface 1100 can connect a perfusion module to the heart 1180. The tissue interface 1100 can physically suspend the heart 1180 roughly in the middle of the cannister, providing for modular connectivity between the heart 1180 and a perfusion module, and facilitate sterile transfer of the heart 1180 from the perfusion system to a sterile surgical environment. The tissue interface 1100 can include multiple ports for connection to the heart 1180, such as the cannulas 1110, 1140, 1150, 1170 and electrode connections 1120, 1160.

The heart 1180 can be connected to the tissue interface 1100 through the coronary sinus sampling cannula 1110, the aortic perfusion cannula 1140, the left atrial perfusion cannula 1150, and the left ventricle balloon catheter 1170

Additional connections between the heart and the cardiac interface may include electrodes (ECG, pacing, defibrillation), ventricular balloon catheters, pressure, flow or temperature sensors, chemical sensors, fluid connections to any of the heart chambers or great vessels, biopsy instruments, strain tensiometers, ultrasonic or doppler instruments, etc.

For example, the first EKG electrode 1120 and the second EKG electrode 1160 can provide for electronic connections. The pass-through 1130 can be for solution movement in and out of the cannister.

For each of the connections between the tissue interface 1100 and the heart 1180, the tissue interface 1100 can be configured to have a matching port or connection point oriented towards the top of the tissue interface 1100 for coupling to a perfusion module. The tissue interface 1100 and attached heart 1180 can be placed into a cannister, such as upon a shelf or ledge designed into the cannister to support and positively locate the cardiac interface.

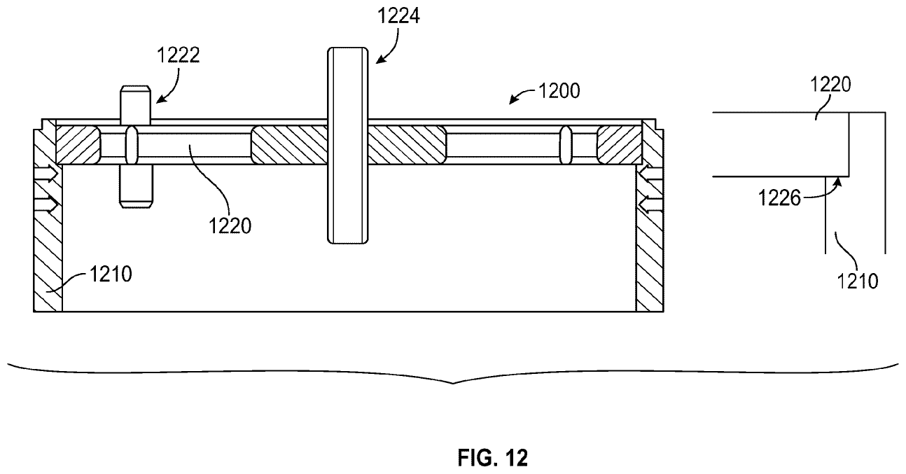

FIG. 12 illustrates a diagram of a perfusion system connector 1200 in an example. The connector 1200 can include a cannister 1210 and a tissue interface 1220 with ports 1222, 1224, and corners 1226.

In connector 1200, the tissue interface 1220 can include top ports 1222, 1224. The top ports 1222, 1224, can connect a heart in the cannister to a perfusion module. The corners 1226 can allow for the tissue interface 1220 to attach to and sit in the cannister.

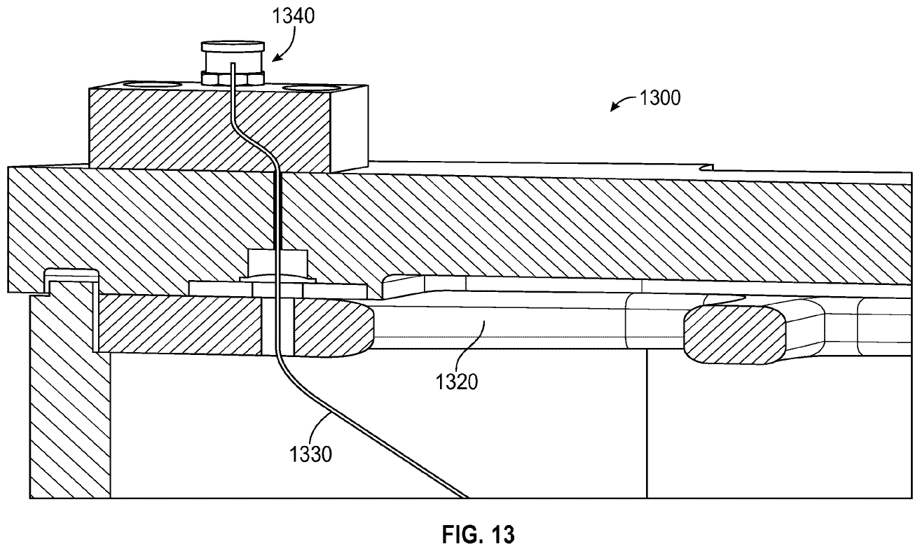

FIG. 13 illustrates a diagram of a perfusion system connector 1300 in an example. The connector 1300 can include a tissue interface 1320, a coronary sinus cannula 1330, and an external port 1340.

Placement of a perfusion module atop a perfusion system connector 1300 can automatically couple ports and connection points of the perfusion module with their matching ports and connection points on the tissue interface 1320. With the perfusion system fully assembled, the heart can be connected via the tissue interface 1320 to the perfusion module.

Some of the connections, such as an aortic cannula, connect to internal components of the perfusion module, such as an oxygenator, pumps, filter, or others. Other connections, such as a coronary sinus catheter, can pass through the tissue interface 1320 to fluid sampling ports for external analysis such as in a blood-gas analysis machine as shown in FIG. 13.

In some examples, electrical connections and signal lines can pass through the tissue interface 1320 for connection to external electrical instruments, such as an ECG, cardiac pacer, defibrillator, or others. In other examples, additional fluid connections may pass through the tissue interface 1320 for connection with external pumps, heat exchangers, oxygenators, or other fluid components.

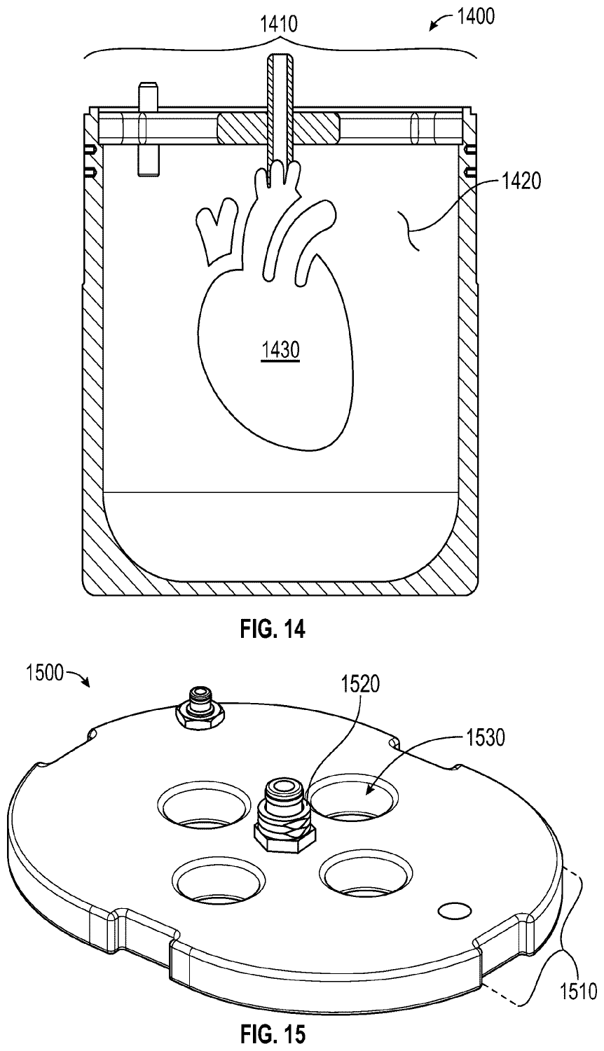

FIG. 14 illustrates a diagram of a perfusion system 1400 in an example. The system 1400 can include a sterile surface 1410, a sterile fluid 1420, and a sterile organ 1430.

Prior to implantation of the heart, the perfusion system 1400 can be disassembled. FIG. 14 shows the perfusion system 1400 without an attached perfusion module. Removal of the perfusion module from the assembly can decouple the perfusion module from the cardiac interface, leaving the cardiac interface and the still-attached heart 1430 suspended within the sterile fluid 1420 and the confines of the cannister. The cardiac interface and heart can be removed singly from the cannister and transferred to a sterile surgical field.

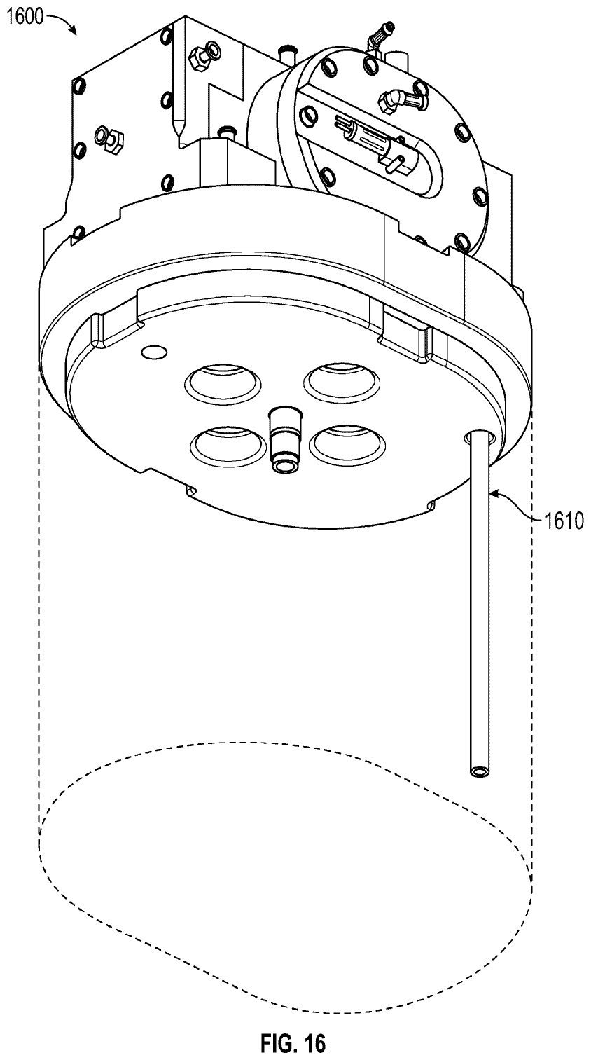

FIG. 15 illustrates a diagram of a perfusion system connector 1500 in an example. The connector 1500 can include a tissue interface 1510, a compression spring 1520 around an aortic coupler, and a grip 1530.

In some cases, the compression spring 1520 or other elastically compressed element disposed between the tissue interface 1510 and a perfusion module can facilitate the separation of the two components when assembly or disassembly is occurring.

Additionally, a grip, such as include finger holes, handles, or other aids for manual manipulation, may be incorporated in the design of the tissue interface 1510 to facilitate lifting the heart out from the cannister and transferring it to a sterile field.

Automatic disconnection or separation of a perfusion module (with its potentially nonsterile external surface) from a sterile cardiac interface can facilitate the sterile transfer of a heart from a perfusion system to a surgical environment. Thus, the tissue interface 1510 and heart can be removed singly from the cannister and transferred to a sterile surgical field.

### Perfusion Fluid Management

FIGS. 16 to 22 below discuss methods and systems for fluid management, such as a perfusate flow management, within a perfusion system.

FIG. 16 illustrates a diagram of a perfusion system 1600 with a dip tube 1610 in an example. The dip tube 1610 can be used to aid in mixing and recirculation of the perfusate within a cannister to avoid areas of stagnation within a perfusate reservoir. The dip tube 1610 can extend towards a bottom of the cannister to draw in perfusate for recirculation through the perfusion module. The dip tube 1610 can be incorporated into or attached to a cardiac interface, or it may be attached directly to a perfusate inlet of the perfusion module and extend down through or past the cardiac interface. The dip tube 1610 may be fenestrated with holes along the side to prevent blockage of flow.

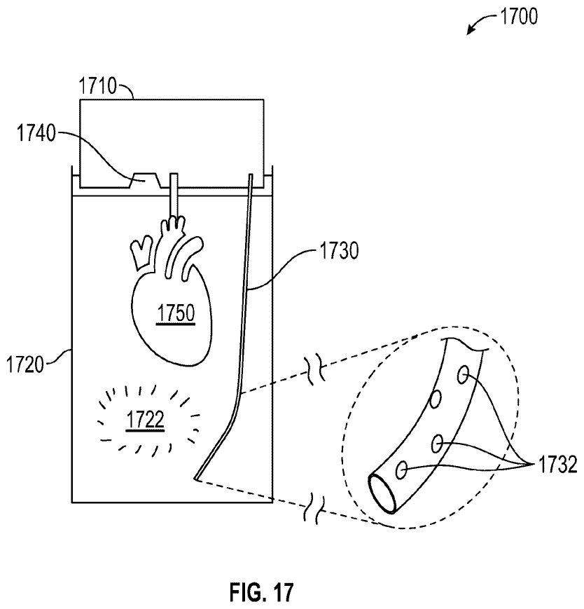

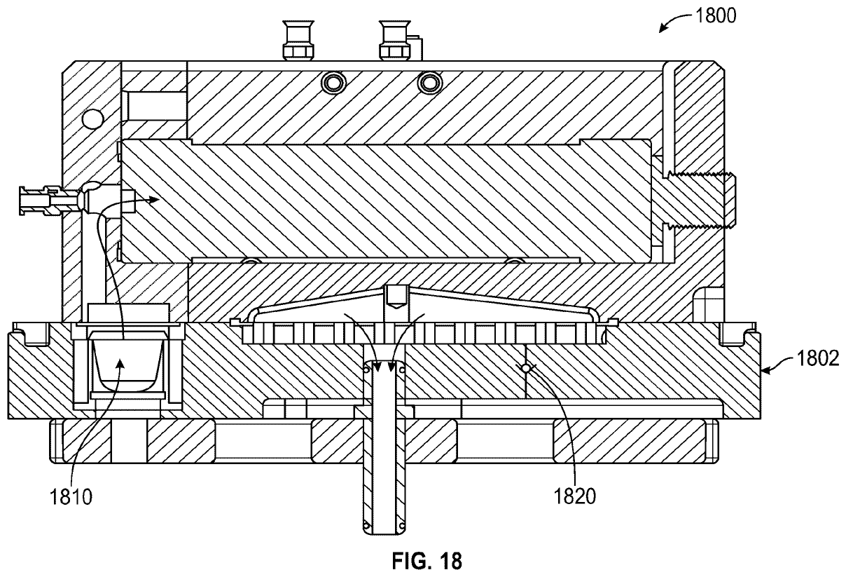

FIG. 17 illustrates a diagram of a perfusion system 1700 in an example. The system 1700 can include a perfusion module 1710, a cannister 1720 with perfusate 1722, a dip tube 1730 with fenestration 1732, and a void space 1740. The system 1700 can host a heart 1750.

Void spaces 1740 or cavities can be incorporated into the underside of the perfusion module 1710 to trap and retain a volume of air to provide a controlled amount of the hydraulic compliance of the system. Some hydraulic compliance can be desired in a closed system in order for diaphragm pumps to function efficiently. The total volume of air in the closed system can help control the hydraulic compliance. Void spaces 1740 or cavities for air retention can range in volume from one cubic centimeter (e.g., least compliant) to 20 cubic centimeters or more (e.g., most compliant).

FIG. 18 illustrates a diagram of a perfusion system 1800 with two one-way valves 1810, 1820 in an example.

In some cases, one-way valve 1810, 1820, can be used in a perfusate return line to aid in the initial priming and setup of the perfusion system 1800. Such one-way valves can be situated, for example, in a dip tube, a cardiac interface, or within a perfusion module upstream of an oxygenator. A one-way valve can prevent retrograde flow of perfusate when perfusate is injected into the perfusion module at any point upstream of pumps. In FIG. 18, a one-way valve 1810 can be positioned within the base plate 1802 of the perfusion module.

In FIG. 18, a second one-way valve 1820 can be situated between the cannister and the filter chamber to aid in initial priming and setup of the perfusion system. During setup, the one-way valve 1820 can allow perfusate to flow upward from the cannister into the filter chamber, thereby filling the chamber from the bottom up with perfusate to displace air via the topmost ports of the filter chamber. During normal operation, the one-way valve 1820 can prevent flow in the reverse direction from the filter directly to the cannister, which would bypass the heart.

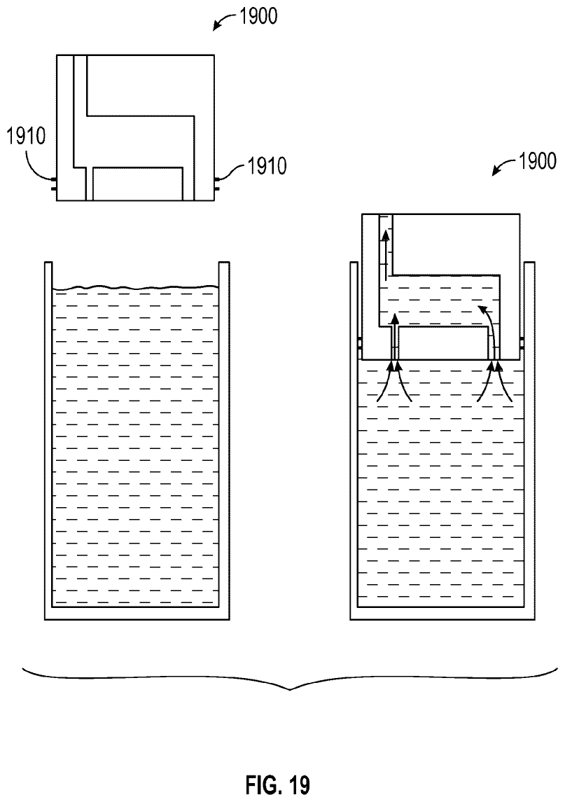

FIG. 19 illustrates a diagram of a perfusion system 1900 with a sliding seal 1910 in an example. Dynamic or sliding seals 1910 can be situated between a perfusion module and cannister such as to facilitate initial priming and setup of the system.

After filling the cannister with perfusate, the insertion of the perfusion module into the cannister can displace the excess perfusate up through the perfusion module, through the return line, dip tube, or one-way valve between cannister and filter chamber. The seals 1910 can be attached to an inner surface of the cannister, an outer side of the perfusion module, or both. In FIG. 19, a generic void space within the perfusion module can be filled up from bottom to top as perfusate is forced into it by the action of pushing the perfusion module into the cannister.

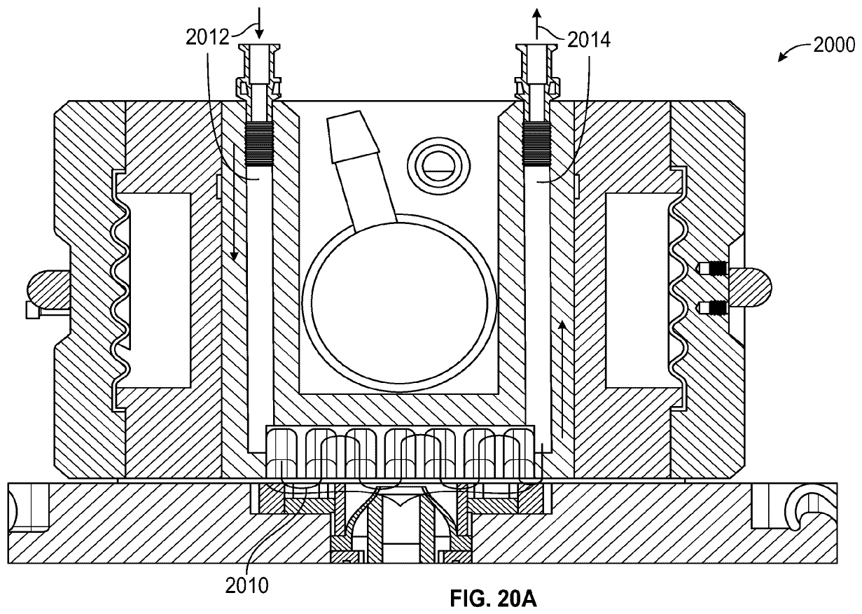

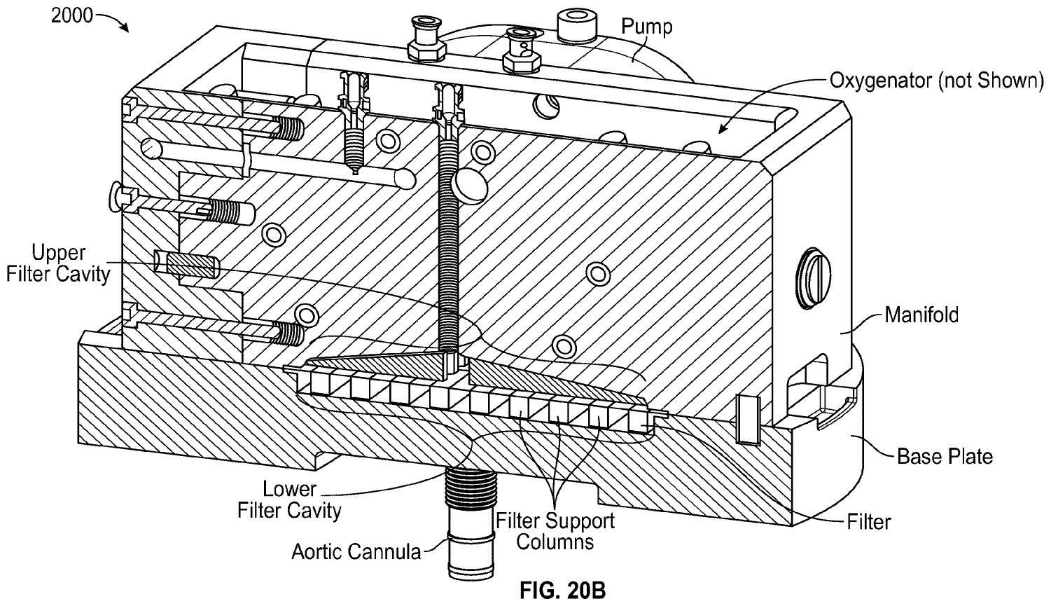

FIG. 20A-20B illustrate diagrams of a perfusion system 2000 two-sided filter chamber 2010 in an example. The system 2000 can include the filter chamber 2010, with filling inlet 2012, venting outlet 2014, upper filler cavity 2016 and lower filter cavity 2018. The system can further include a pump 2020, an oxygenator 2030, a manifold 2040, a base plate 2050, and a cannula 2060. In system 2000, a two-sided filter chamber design can facilitate initial liquid priming of the aortic cannula 2060 and filter chamber, and purging of air from the same spaces.

In some cases, the filter chamber 2010 and aortic cannula can be filled from the top down, from the pump, or from a top vent in the system. The two-sided design of the filter chamber 2010 can allow filling from one side and venting of air from the other side, without having the conflict of downward traveling liquid and upward traveling gas in the same conduit. The cross-sectional view shows the two conduits leading fluid down from the pump outlets to the filter chamber, each conduit having a filling or purging port at the top.

The location and design of the filter chamber 2010 can facilitate manufacturing and assembly of the filtering elements of the perfusion module. The filter can be held in place between the base plate 2050 of the perfusion module, and a manifold 2040 that holds the oxygenator and pumps. The cavity within the manifold 2040 can spread the otherwise narrow stream of perfusate across the wide area of the filter, and the cavity within the base plate 2030 can narrow the wide filter stream back into a single narrow stream for connection to the aortic cannula 2060. Ribs or columns within the base plate 2030 cavity can support the filter against distention by hydraulic pressure without substantially blocking the filter area. In some cases, the filter can a porous metal screen having pore size ranging from about 20 to 40 microns. In other cases, the filter can include a polymeric mesh, felt, screen, or other filter media.

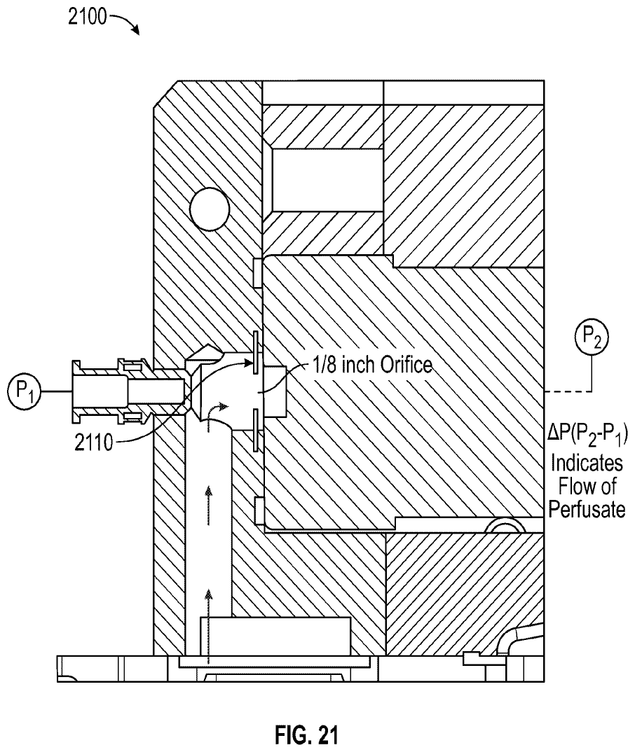

FIG. 21 illustrates a diagram of a perfusion system 2100 with an orifice restrictor 2110 in an example. The orifice restrictor 2110 can be in an oxygenator inlet and can be used to facilitate measurement of perfusate flow. The orifice restrictor 2110, combined with a known fluid resistance of the oxygenator, can provide for a pressure differential that can be linearly correlated to the flow rate of perfusate through the system 2100. A pressure differential sensor can thus be used to indicate flow of perfusate through the system 2100. In an example, orifice plates with a single hole ranging from about 1/64 inch to 3/16-inch diameter, placed immediately upstream of the oxygenator, can provide for a pressure drop signal that can be used to indicate perfusate flow in the range of about 20 cc/min to 250 cc/min.

In another example, the orifice restrictor can be placed elsewhere in the fluid path (baseplate or manifold of the perfusion module (102 and 101 of FIG. 1, respectively)

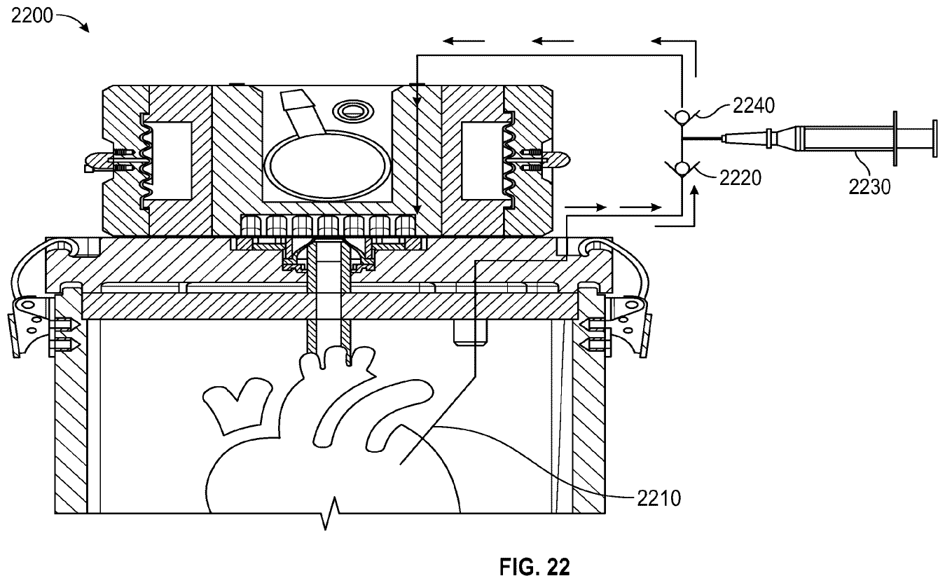

FIG. 22 illustrates a diagram of a perfusion system 2200 with a coronary sinus sample recirculation loop 2210 in an example. A coronary sinus sample recirculation loop 2210 can facilitate priming of the coronary sinus sample tube with fresh venous perfusate from the heart without wasting the priming volume of the tubing.

For example, fluid samples of about 100 microliters to 1 milliliter may be desired from the coronary sinus. However, the volume of the sample tubing between the coronary sinus and the sample port may be several milliliters or more. Priming of the sample lines to obtain a fresh fluid sample from the coronary sinus can entail the extraction of several milliliters “priming volume” of perfusate from the sample tubing, before the fresh sample is obtained.

The recirculation loop 2210 can be provided to allow that the priming volume can be directly returned to the perfusion system 2200 to avoid depletion of the total perfusate volume. Situated between the coronary sinus sample line and the syringe port can be a first one-way valve 2220. The first one-way valve 2220 can allow for sample fluid to be drawn from the coronary sinus towards the syringe 2230 but not in the opposite direction.

Also connected to the syringe port can be a second one-way valve 2240, having a fluid return line back to the perfusion system connected to the cannister or to the perfusion module. The second one-way valve 2240 can allow passage of fluid from the syringe 2230 to the perfusion system (e.g., cannister or perfusion module), but not in the opposite direction.

The resultant sample loop 2210 therefore can operate as follows: (1) a sterile sample syringe 2230 can be attached to the sample port; (2) withdrawal of the syringe plunger can pull fresh sample fluid from the coronary sinus into the sample line; (3) the fluid pulled into the syringe can include at least some, if not all of the priming volume; (4) depression of the syringe plunger can push the priming volume back into the perfusion system; and (5) a second withdrawal of the syringe plunger can pull a second volume from the sample line.

The volume pulled from the sample line into the syringe can be the same fresh coronary sinus fluid sample that was pulled from the coronary sinus into the sample line by step 2 above. Using this method, a fresh fluid sample can be extracted for analysis without wasting the 2 ml priming volume. In other examples, the plunger of the sampling syringe may be cycled more than two times to fully prime the sample line with fresh sample fluid.

In another embodiment, a secondary pump may be used to continuously refresh the sample loop with fluid from the coronary sinus, eliminating the need to use the syringe 2230 to prime the sample loop.

### Perfusion Thermal Regulation

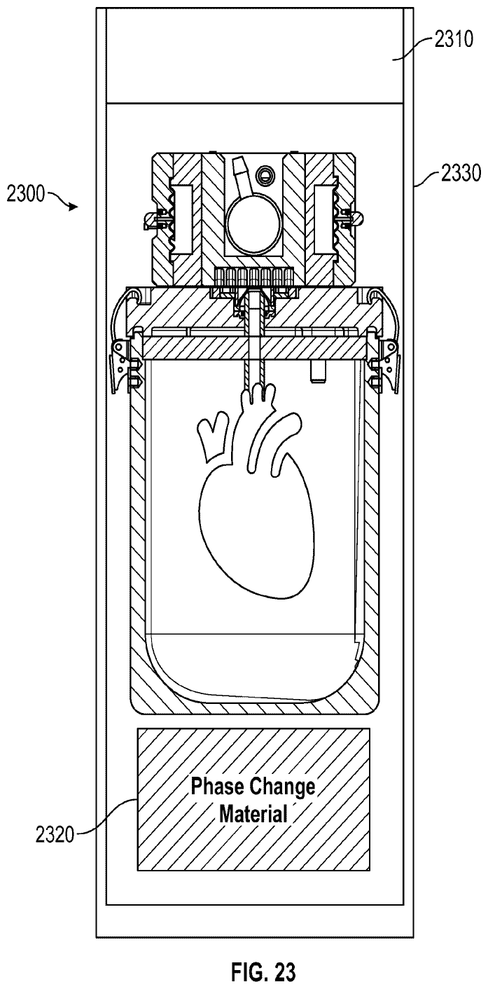

FIG. 23 illustrates a diagram of a perfusion system 2300 with thermal regulation in an example. The system 2300 can include a foam top 2310, a phase change material 2320, and a vacuum insulated container 2330.

Thermal regulation of the perfusion system can be helpful to maintain hypothermic conditions over time. For example, after initiating perfusion at a typical system temperature of about 4-15° C., the perfusion system can be enclosed within a thermal barrier to prevent or retard the warming of the system.

In the system 2300, a foam top 2310 can be used as such a thermal barrier. In some cases, the thermal barrier may comprise polymer foam, vacuum insulated panels, aerogel, vacuum insulated Dewar vessels, or any combination of these

Temperature sensitivity of the heart (or other organ or tissue), and the perfusion system 2300 may also desire the inclusion of a phase change material 2320, such as water, ice, or Puretemp 4 (Entropy Solutions Inc., Plymouth MN). Such a phase change material can be placed inside the thermal barrier, such as vacuum insulated container 2330, to stabilize the temperature of the perfusion system 2300 over the desired time interval. The phase change material 2320 can be enclosed within one or more fluid-tight containers or compartments to prevent leakage of melted water or materials.

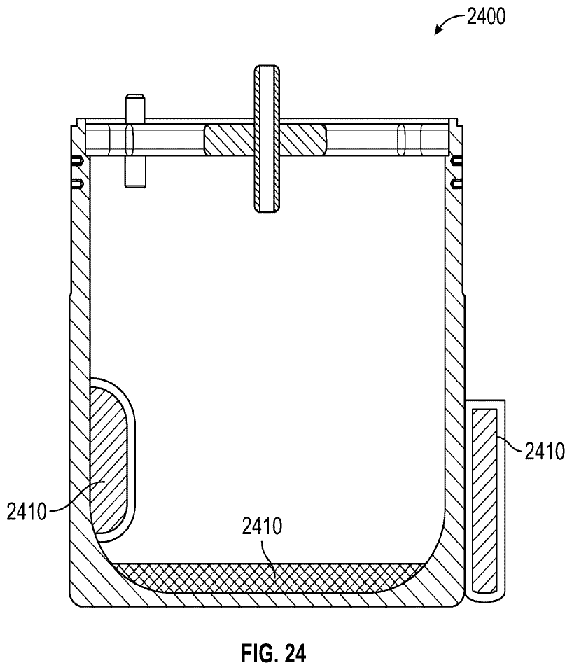

FIG. 24 illustrates a diagram of a perfusion system 2400 with phase change materials 2410 in an example. In the system 2400, the phase change materials 2410 can be incorporated into the design of the cannister. In some cases, the cannister can hold a captive volume of phase change material, separated from the perfusate reservoir by a fluid-tight barrier. For example, pre-freezing of the cannister and its pre-loaded phase change material can help for easy packing of the cold components, such as the perfusion system 2400 plus phase change materials 2410, within the thermal barrier and facilitates heat transfer between the perfusate and the phase change material 2410 by reducing air gaps that may otherwise limit heat flow under loose packing conditions.

### Perfusion Oxygen Flow

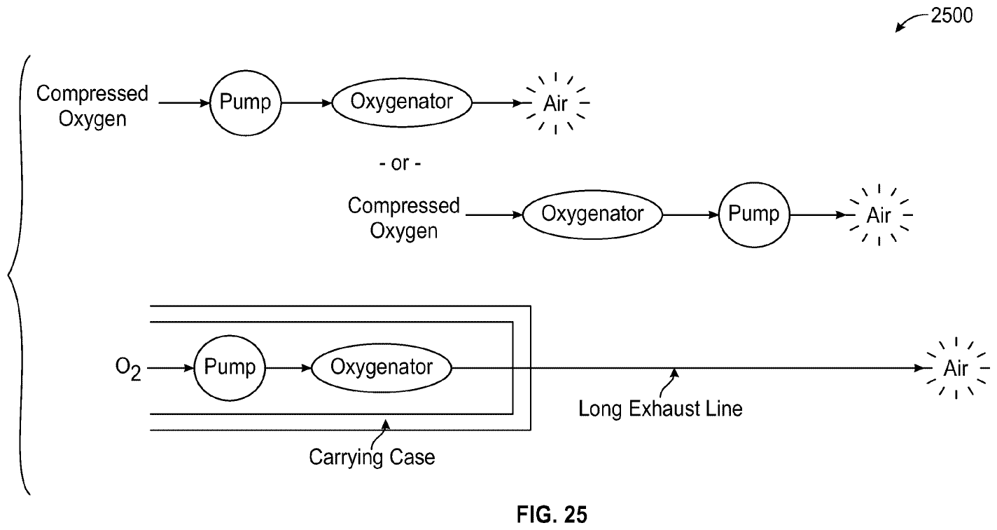

FIGS. 25 to 28B below show and discuss alternative fluid flow paths within a perfusion system.

FIG. 25 illustrates a diagram of a perfusion system 2500 oxygen flow path in an example. The system 2500 can use compressed oxygen gas to first supply mechanical energy to the diaphragm pumps and then to oxygenate the perfusate in the oxygenator before being vented to the surrounding atmosphere. Alternatively, the oxygen may first flow through the oxygenator and then to the pumps, to be discharged by the pumps to the surrounding atmosphere.

In some cases, the oxygen can pass from only one pump into the oxygenator, and the oxygen from the second pump can be exhausted directly to the surrounding atmosphere. This configuration can minimize pump exhaust pressure and can increase overall pump efficiency.

In other examples, after passing through the pump and oxygenator, the oxygen can be channeled away from the perfusion system by an exhaust line, terminating outside an external carrying case. This configuration can limit oxygen-enrichment of the space immediately surrounding the perfusion system and its electronics.

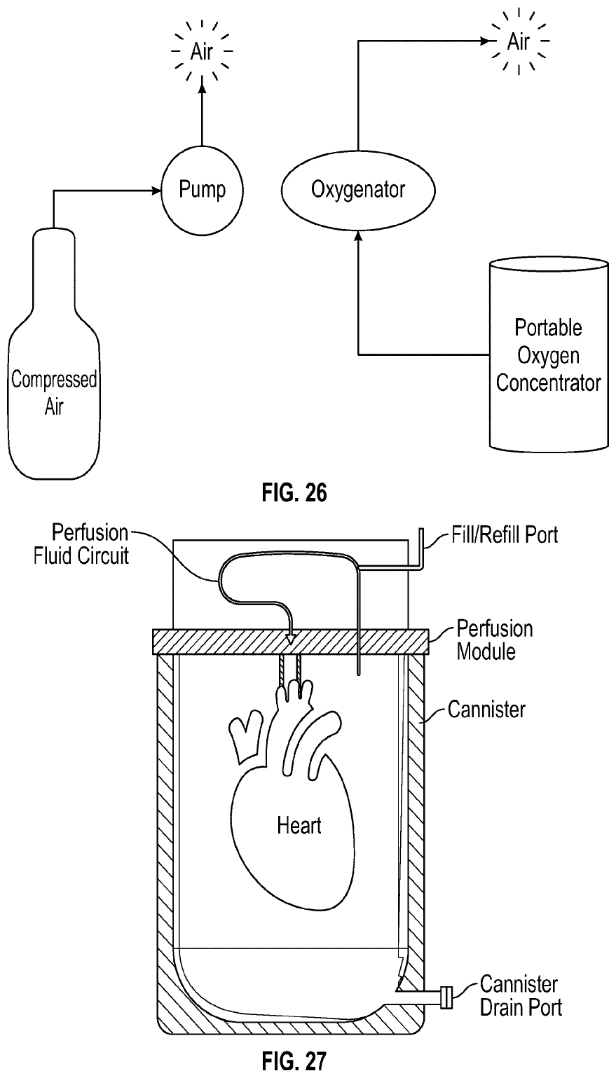

FIG. 26 illustrates a diagram of a perfusion system oxygen flow path in an example. Under some conditions, oxygen may be limited to use only as a supply to the oxygenator for gas exchange with the perfusate, and the pumps may be powered by compressed nitrogen, air, other gases, or electrical energy. The amount of oxygen required by the oxygenator alone can be less than the amount required to actuate the diaphragm pumps. The small volume of oxygen (e.g., about 10 liters STP) used exclusively for non-pumping purposes can be provided by a small and/or medium pressure oxygen tank, or a chemical reaction (e.g., catalytically controlled hydrogen peroxide decomposition), or a water electrolyzing electrochemical cell, or a portable oxygen concentrator.

FIG. 27 illustrates a diagram of a perfusion system in an example. An exchange of new perfusate for used perfusate may be desired after a period of continuous perfusion (e.g., about 10 hours or more), to replace metabolic substrates or to remove accumulated metabolites from the system. Alternatively, it may be advantageous to change from one type of perfusate to another type of perfusate as the system transitions from hypothermic temperatures to normothermic (physiologic) temperatures. Solution changes can be facilitated by fill ports and drain ports in the perfusion system. The ports may be located at the bottom of the cannister, or in connection with the fluid circuit in the perfusion module.

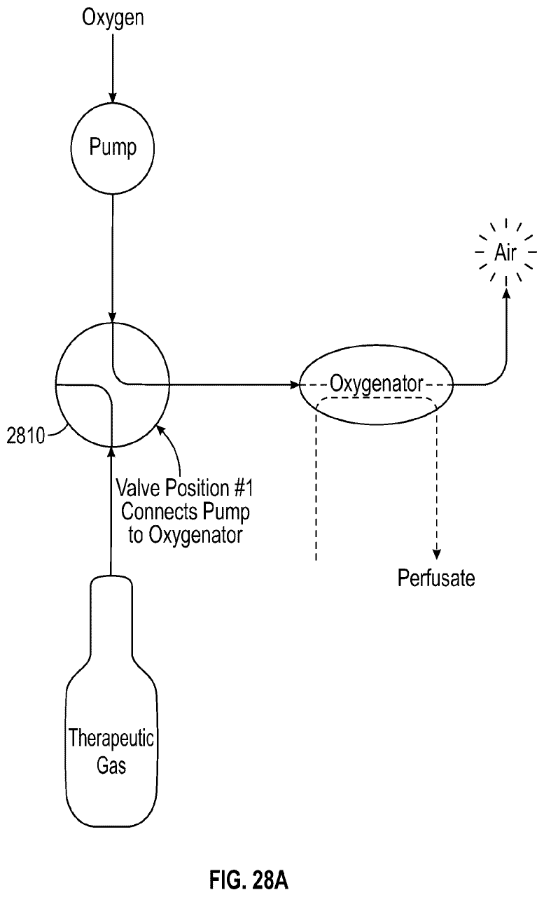

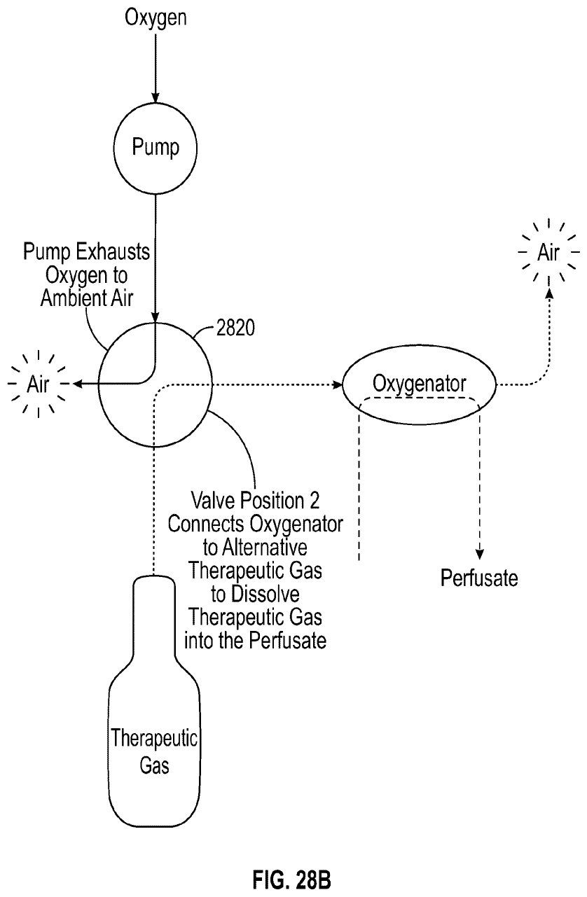

FIGS. 28A-28B illustrate diagrams of a perfusion system 2800 having alternative therapeutic gases in an example. FIG. 28A depicts the system 2800 with a gas diverter valve 2810 in a first position, for normal operation, wherein therapeutic gas is not flowing. FIG. 28B depicts the system 2800 with the gas diverter valve 2820 in a second position, diverting oxygen from the pump directly to the air, and routing the therapeutic gas to the oxygenator.

At some phases of the organ care process, a therapeutic gas other than oxygen can be used to treat the organ. By incorporation of a switching valve 2820 in the oxygen gas line between the pumps and the oxygenator, alternative therapeutic gases can be dissolved into the perfusate using the oxygenator as a gas exchanger. Therapeutic gases such as carbon monoxide, nitrous oxide, hydrogen sulfide, hydrogen, or others may be temporarily administered to the organ by dissolving those gases into the perfusate.

In one example, therapeutic gas can be administered substantially simultaneously, alternatively, or in an overlapping fashion with oxygenating the perfusate fluid. Overlapping fashion includes receiving at least a portion of the therapeutic gas at the same time as a portion of the oxygen is received.

In another example, a mixture of gases can be administered from a single tank of premixed, compressed gas. Pre-mixed gas tanks are available that may be used, such as a tank containing a small percentage of hydrogen. One example premixed tank includes 3% hydrogen and 97% oxygen.

### VARIOUS NOTES & EXAMPLES

1. A perfusion system includes a perfusion module configured to circulate a perfusate. The perfusion module includes an oxygenator configured to oxygenate the perfusate, the oxygenator configured to be fluidly coupled with an oxygen source and configured to receive oxygen therefrom, a first pump operably coupled with the oxygenator and configured to circulate the perfusate through the oxygenator, and a second pump operably coupled with the oxygenator and configured to circulate the perfusate through the oxygenator. A cannister has a receptacle to receive target tissue. A tissue interface is disposed between the perfusion module and the cannister. The tissue interface includes at least one port for mechanically and fluidically coupling the target tissue to the perfusion module.
2. The perfusion system of example 1, wherein at least one of the first and second pumps are diaphragm pumps.
3. The perfusion system of any of examples 1-2, wherein at least one of the first and second pumps each include a pump body, a pump cover configured to fit over the pump body, and a diaphragm that includes an elastic material, the diaphragm situated between the pump body and the pump cover.
4. The perfusion system of example 3, further including a solenoid valve configured to actuate the diaphragm.
5. The perfusion system of any of examples 3-4, further including an inlet and an outlet configured for movement of perfusate fluid therethrough responsive to movement of the diaphragm.
6. The perfusion system of any of examples 3-5, further including a first set of convolutions on the diaphragm and a second set of convolutions on the pump cover, wherein the first set of convolutions and the second set of convolutions mate with each other.
7. The perfusion system of example 6, wherein the first and second sets of convolutions comprise nested racetrack shaped, oval, or circular convolutions.
8. The perfusion system of any of examples 1-7, wherein the first pump and the second pump are connected in parallel.
9. The perfusion system of any of examples 1-8, wherein the first pump and the second pump are connected in series.
10. The perfusion system of any of examples 1-9, wherein the first pump and the second pump are configured to operate on duty cycles that are in opposing phases.
11. The perfusion system of any of examples 1-10, wherein the first pump and the second pump are configured to operate on duty cycles that are 180 degrees apart.
12. The perfusion system of any of examples 1-11, wherein the first pump and the second pump are configured to operate in an alternating fashion.
13. The perfusion system of any of examples 1-12, further comprising a pressure regulator fluidly coupled to the oxygen source.
14. The perfusion system of example 13, further comprising a flow 1 restricting element downstream of the pressure regulator.
15. The perfusion system of any of examples 13-14, further comprising an accumulator downstream of the flow restricting element.
16. The perfusion system of any of examples 1-15, wherein the tissue interface comprises a plurality of ports.
17. The perfusion system of example 16, wherein one of the ports comprises a cannula configured to fluidly couple tissue to the perfusion module.
18. The perfusion system of any of examples 16-17, wherein one of the plurality of ports include a catheter configured to fluidly couple tissue to the perfusion module.
19. The perfusion system of any of examples 16-18, wherein one of the plurality of ports include an electrode configured to electrically couple tissue to the perfusion module.
20. The perfusion system of any of examples 16-19, wherein one of the plurality of ports include a balloon catheter.
21. The perfusion system of any of examples 16-20, wherein at least one of the plurality of ports include an aortic perfusion cannula, a left atrial perfusion cannula, a left ventricle cannula, or a coronary sinus sampling cannula.
22. The perfusion system of any of examples 16-21, wherein one of the plurality of ports include a pass-through fluidly coupled to an internal portion of the cannister.
23. The perfusion system of any of examples 1-22, wherein the cannister includes one or more corners for mechanical connection of the tissue interface to the cannister.
24. The perfusion system of any of examples 1-23, wherein the tissue interface further includes a compression spring for attachment to the perfusion module.
25. The perfusion system of any of examples 1-24 wherein the tissue interface further includes a grip for manual manipulation.
26. The perfusion system of example 25, wherein the grip includes finger holes.
27. The perfusion system of any of examples 1-26, wherein the tissue interface further includes a dip tube configured to extend into the cannister.
28. The perfusion system of any of examples 1-27, wherein an underside of the perfusion module includes one or more void spaces.
29. The perfusion system of any of examples 1-28, wherein the perfusion module further includes a one-way valve configured to allow flow of perfusate from the cannister to the perfusion module.
30. The perfusion system of any of examples 1-29, wherein the perfusion module further includes a one-way valve configured to allow priming of the cannister with perfusate.
31. The perfusion system of any of examples 1-30, wherein the perfusion module further includes one or more sliding seals for engaging the perfusion module with the cannister.
32. The perfusion system of any of examples 1-31, wherein the perfusion module further includes a multi-compartment filter chamber.
33. The perfusion system of any of examples 1-32, wherein the perfusion module further includes a sampling line.
34. The perfusion system of any of examples 1-33, further comprising a thermal insulating material at least partially encasing the perfusion module and the cannister.
35. The perfusion system of example 34 wherein the thermal insulating material includes a vacuum-insulated dewar vessel.
36. The perfusion system of any of examples 1-35, further including a carrying case at least partially encasing the perfusion module, the tissue interface, and the cannister.
37. The perfusion system of any of examples 1-36, further including a portable oxygen source fluidly coupled to the oxygenator.
38. The perfusion system of any of examples 1-37, further including a power source electrically coupled to the perfusion module.
39. The perfusion system of any of examples 1-38, further including a gas diverter valve fluidly coupled to the oxygen source, upstream of the oxygenator.
40. The perfusion system of any of examples 1-39, further including a therapeutic gas source coupled to the oxygenator.
41. The perfusion system of example 39, wherein the therapeutic gas source includes carbon monoxide, nitrous oxide, hydrogen sulfide, hydrogen, or combinations thereof.
42. A method of perfusing target tissue in a perfusion system, the method including oxygenating perfusate in an oxygenator, pumping the oxygenated perfusate from the oxygenator to a de-pressurized pump chamber, pressurizing the pump chamber and pumping the oxygenated perfusate out of the pump chamber via a first pump and a second pump and through a cannula to the target tissue in a cannister, and oxygenating the target tissue and de-oxygenating the perfusate.
43. The method of example 42, wherein pumping the oxygenated perfusate includes alternately pumping through the first pump and the second pump.
44. The method of any of examples 42-43, wherein pumping the oxygenated perfusate includes alternately pumping through the first pump and the second pump in opposite duty cycles.
45. The method of any of examples 42-44, further including closing an aortic valve.
46. The method of any of examples 42-45, further including introducing a bolus into the perfusion system prior settling into a lower target oxygenated perfusate flow rate.
47. The method of any of examples 42-46, further including gradually decreasing a stroke rate of the pumping.
48. The method of any of examples 42-47, further including gradually increasing a vent time of the pumping.
49. The method of any of examples 42-48, further including gradually decreasing pressurizing time.
50. The method of any of examples 42-49, further including sampling the perfusate.
51. The method of any of examples 42-50, further including priming the perfusion system.
52. The method of any of examples 42-51, further including maintaining the tissue in a sterile cannister coupled to the perfusion system.
53. The method of any of examples 42-52, further including attaching a tissue interface to a perfusion module in the perfusion system.
54. The method of example 53, further including detaching the tissue interface from the perfusion module in the system using a compression spring.
55. The method of any of examples 42-54, further including introducing a therapeutic gas into the perfusion system.
56. The method of example 55, wherein introducing the therapeutic gas is performed alternatively, simultaneously, or in an overlapping fashion with oxygenating the perfusate fluid.
57. The method of any of examples 42-56, wherein pumping includes pressurizing the perfusion system with oxygen flow.
58. The method of any of examples 42-57, further including venting excess oxygen into atmosphere.
59. The method of any of examples 42-58, further including thermally protecting the perfusion system.
60. The method of example 59, wherein thermally protecting the perfusion system includes applying a phase change material to at least a portion of the cannister.
61. A method of priming the perfusate system of example 1 including filling the canister with perfusate and sliding the perfusion module to overlap with a top portion of the cannister with sliding seals disposed between the perfusion module and the canister to prime the perfusate system.
62. The method of example 61 wherein excess perfusate is displaced up through the perfusion module.
63. The method of example 62 wherein the perfusate is displaced through a return line, dip tube, or one-way valve between cannister and a filter chamber of the perfusion module.
64. The method of any of examples 61-63 wherein the sliding seals are attached to an inner surface of the cannister, an outer surface of the perfusion module, or both the inner surface of the cannister and outer surface of the perfusion module.
65. The method of any of examples 61-64 wherein a generic void space within the perfusion module can be filled up from bottom to top as perfusate is forced into it by the action of sliding the perfusion module.

Each of these non-limiting examples can stand on its own or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A perfusion system comprising:
a perfusion module configured to circulate a perfusate, the perfusion module comprising:
an oxygenator configured to oxygenate the perfusate, the oxygenator configured to be fluidly coupled with an oxygen source and configured to receive oxygen therefrom;
a first pump operably coupled with the oxygenator and configured to circulate the perfusate through the oxygenator; and
a second pump operably coupled with the oxygenator and configured to circulate the perfusate through the oxygenator;
a cannister having a receptacle to receive target tissue; and
a tissue interface disposed between the perfusion module and the cannister, the tissue interface including at least one port for mechanically and fluidically coupling the target tissue to the perfusion module.

2. The perfusion system of claim 1, wherein at least one of the first and second pumps comprise diaphragm pumps.

3. The perfusion system of claim 1, wherein at least one of the first and second pumps each comprise: a pump body; a pump cover configured to fit over the pump body; and a diaphragm comprising an elastic material, the diaphragm situated between the pump body and the pump cover.

4. The perfusion system of claim 3, further comprising at least one of:
a solenoid valve configured to actuate the diaphragm; or
a first set of convolutions on the diaphragm and a second set of convolutions on the pump cover, wherein the first set of convolutions and the second set of convolutions mate with each other.

5. The perfusion system of claim 1, wherein the first pump and the second pump are connected in parallel or in series.

6. The perfusion system of claim 1, wherein the first pump and the second pump are configured to operate on duty cycles that are in opposing phases or in an alternating fashion.

7. The perfusion system of claim 1, further comprising:
a pressure regulator fluidly coupled to the oxygen source;
a flow restricting element downstream of the pressure regulator; and
an accumulator downstream of the flow restricting element.

8. The perfusion system of claim 1, wherein the tissue interface comprises a plurality of ports, at least one of the plurality of ports comprising:
a cannula configured to fluidly couple tissue to the perfusion module, the cannula comprising one of an aortic perfusion cannula, a left atrial perfusion cannula, a left ventricle cannula, or a coronary sinus sampling cannula.

9. The perfusion system of claim 1, wherein the tissue interface further comprises a grip for manual manipulation.

10. The perfusion system of claim 1, wherein the perfusion module further comprises at least one of:
a one-way valve configured to allow priming of the cannister with perfusate; or
one or more sliding seals for engaging the perfusion module with the cannister.

11. The perfusion system of claim 1, further comprising:
a gas diverter valve fluidly coupled to the oxygen source, upstream of the oxygenator.

12. The perfusion system of claim 1, further comprising:
a therapeutic gas source coupled to the oxygenator, the therapeutic gas source including carbon monoxide, nitrous oxide, hydrogen sulfide, hydrogen, or combinations thereof.

13. A method of perfusing target tissue in a perfusion system, the method comprising:
oxygenating perfusate in an oxygenator;
pumping the oxygenated perfusate from the oxygenator to a de-pressurized pump chamber;
pressurizing the pump chamber and pumping the oxygenated perfusate out of the pump chamber via a first pump and a second pump and through a cannula to the target tissue in a cannister; and
oxygenating the target tissue and de-oxygenating the perfusate.

14. The method of claim 13, wherein pumping the oxygenated perfusate comprises alternately pumping through the first pump and the second pump in parallel or pressurizing the perfusion system with oxygen flow.

15. The method of claim 13, further comprising at least one of:
introducing a bolus into the perfusion system prior to settling into a lower target oxygenated perfusate flow rate;
regulating a duty cycle of the pumping by at least one of decreasing a stroke rate of the pumping, increasing a vent time of the pumping, or decreasing pressurizing time; or
introducing a therapeutic gas into the perfusion system, wherein introducing the therapeutic gas is performed alternatively, simultaneously, or in an overlapping fashion with oxygenating the perfusate.

16. A perfusion system comprising:
an oxygenator configured to oxygenate a perfusate;
a first pump operably coupled to the oxygenator and configured to circulate the perfusate through the oxygenator;
a second pump operably coupled to the oxygenator and configured to circulate the perfusate through the oxygenator;
a cannister having a receptacle to receive target tissue; and
a tissue interface including at least one port for mechanically and fluidically coupling the target tissue to receive the perfusate.

17. The perfusion system of claim 16, wherein at least one of the first and second pumps each comprise:
a pump body;
a pump cover configured to fit over the pump body; and
a diaphragm comprising an elastic material, the diaphragm situated between the pump body and the pump cover.

18. The perfusion system of claim 17, further comprising at least one of:
a solenoid valve configured to actuate the diaphragm; or
first set of convolutions on the diaphragm and a second set of convolutions on the pump cover, wherein the first set of convolutions and the second set of convolutions mate with each other.

19. The perfusion system of claim 16, wherein the first pump and the second pump are connected in parallel or in series.

20. The perfusion system of claim 16, wherein the first pump and the second pump are configured to operate on duty cycles that are in opposing phases; or in an alternating fashion.
